(19) United States
(12) Patent Application Publication
(10) Pub. No.: US 2026/0286322 A1
(43) Pub. Date: Sep. 24, 2026

(54) PROTEIN TYROSINE PHOSPHATASE WEDGE DOMAIN PEPTIDE DIMER FOR NERVOUS SYSTEM REPAIR

(71) Applicant: NervGen Pharma Corp., Vancouver (CA)

(72) Inventor: Matvey LUKASHEV, Upton, MA (US)

(21) Appl. No.: 19/168,200

(22) PCT Filed: Apr. 3, 2024
(86) PCT No.: PCT/IB2024/053264

§ 371 Date: Sep. 23, 2025

Related U.S. Application Data
(60) Provisional application No. 63/493,912, filed on Apr. 3, 2023.

(51) Int. Cl. C12N 9/16; A61K 38/00; A61P 25/28

(52) U.S. Cl. CPC C12N 9/16; A61P 25/28; A61K 38/00; C07K 2319/10; C12Y 301/03048

(57) ABSTRACT

The disclosure relates to compositions and methods for nervous system repair. The compositions comprise a peptide that is a dimer, which comprises a first monomer covalently cross-linked with a second monomer, each monomer comprising a domain comprising an amino acid sequence derived from a cytoplasmic wedge domain of a receptor-type protein-tyrosine phosphatase (PTPR). The disclosure also relates to methods and compositions for treating diseases, disorders, and/or conditions associated with inhibition of nervous system repair by chondroitin sulfate proteoglycans (CSPG) or with suppressive effects of LAR family phosphatases, such as PTPRD, PTPRF, and PTPRS on neuro repair.

### RELATED APPLICATIONS

This application claims priority to U.S. Provisional Application No. 63/493,912 filed on Apr. 3, 2023, the entire contents of which are hereby incorporated by reference in their entirety.

### REFERENCE TO SEQUENCE LISTING

The instant application contains a Sequence Listing which has been submitted electronically in XML format and is hereby incorporated by reference in its entirety. Said XML copy, created on Mar. 21, 2024, is named 753221 NGT-001 PC.xml and is 17,882 bytes in size.

### BACKGROUND

Spinal cord injury and other conditions associated with central (CNS), or peripheral (PNS) nervous system damage can cause permanent disability or loss of motor, sensory and/or cognitive function. Recovery after CNS injury is limited, leading to substantial current interest in potential strategies to overcome this challenge. A fundamental obstacle facing efforts to improve neuronal function after injury is the inability of the adult CNS to regenerate.

Two well-known classes of regeneration inhibitors are myelin-associated inhibitors (e.g., MAG, Nogo and OMGP) and inhibitors in scar tissue formed by glia at the injury site (e.g., chondroitin sulfate proteoglycans (CSPGs)). CSPG deposition causing inhibition of axonal and synaptic repair at sites of nervous system damage has been implicated as a key contributor not only to the pathogenesis of traumatic CNS injury, but to the progression of number of neurodegenerative and neuroinflammatory diseases as well.

CSPGs present a barrier to axon regeneration via several inhibitory mechanisms. The inhibitory effects of CSPG are not only reflected in the formation of dystrophic axonal retraction bulbs that fail to regenerate through the lesion, but also in the limited ability for collateral sprouting of spared fibers. Although it has been known for nearly two decades that sulfated proteoglycans are major contributors to the repulsive nature of the glial scar, the precise inhibitory mechanism was poorly understood.

Protein tyrosine phosphatases (PTPs) play an important role during dephosphorylation, a process that can remove phosphoryl groups from phosphotyrosine-containing proteins (Jing-Fei Huang, Molecular Biology and Evolution, Volume 20, Issue 5, May 2003, Pages 815-820). Receptor-type protein tyrosine phosphatases (PTPRs) are a subgroup of PTPs that share a transmembrane domain with resulting similarities in function and target specificity (Du Y, Grandis JR. Chin J Cancer. 2015; 34(2):61-69).

The leukocyte common antigen related (LAR) subfamily PTPRs consists of three members: LAR (PTPRF), receptor protein tyrosine phosphatase sigma (PTPRS) and receptor protein tyrosine phosphatase delta (PTPD). PTPRS and PTPF have been identified as receptors for CSPGs, the principal inhibitory constituents of the glial scar and perineuronal net. The sugar side chains of CSPGs can bind to PTPRF and PTPRS expressed by cells, such as neural cells, and inhibit neural cell growth, plasticity, regeneration and sprouting failure in the neural cells.

It was found that PTPRS-deficient neurons exhibit decreased sensitivity to CSPG-mediated inhibition in various cell-based assays and showed increased regeneration following neurological injury, such as following spinal cord injury and optic nerve crush. The results in the PTPRF knockout remained inconclusive, with both increased and decreased regenerative phenotypes being found. Since CSPGs are the primary impediment to regeneration and plasticity in the injured adult nervous system, modulators of the LAR family protein tyrosine phosphatase functions can be used as therapeutic agents promoting neural plasticity, regeneration and ultimately repair of nervous system damage.

In view of the foregoing, there remains an urgent need for compositions that modulate and attenuate the inhibitory CSPG function. In addition, there remains a need for compositions that can alleviate CSPG-induced cellular and neurologic deficits associated with the function of the LAR family protein tyrosine phosphatases.

### SUMMARY

The present disclosure provides wedge domain peptide dimers for nervous system repair and treatment of neurologic deficits associated with the function of the LAR family protein tyrosine phosphatases. The wedge domain peptide dimers are thus useful in the treatment of diseases, disorders, and/or conditions associated with CSPG-mediated suppression of nervous system repair.

The wedge domain peptide dimers are also useful for treating associated diseases and disorders in a subject in need thereof.

In an aspect, the application pertains to a pharmaceutical composition comprising a peptide wherein the peptide is a dimer comprising a first monomer covalently cross-linked with a second monomer, each monomer comprising a domain comprising an amino acid sequence derived from a cytoplasmic wedge domain of a receptor-type protein-tyrosine phosphatase (PTPR); and wherein the mass ratio of the cross-linked dimer to free monomer in the pharmaceutical composition is greater than 1:20.

In an aspect, the application pertains to a pharmaceutical composition comprising a peptide dimer, where the peptide dimer comprises an amino acid sequence comprising two monomer subunits, each monomer subunit comprising a peptide domain comprising an amino acid sequence independently selected from the group consisting of a PTPRF wedge domain, a PTPRD wedge domain, and a PTPRS wedge domain, and variants having at least 70% identity thereto; and wherein the first monomer subunit is bound to the second monomer subunit.

In another aspect, provided herein is a method of treating a neurological condition, disease or disorder selected from the group consisting of neural injury, neurological inflammatory or autoimmune disease, and neurodegenerative disease in a subject in need thereof, the method comprising administering an effective amount of a pharmaceutical composition to the subject, wherein the pharmaceutical composition comprises a peptide dimer or pharmaceutically acceptable salt or solvate thereof, wherein the peptide domain comprises an amino acid sequence independently selected from the group consisting of a PTPRF wedge domain, a PTPRD wedge domain, and a PTPRS wedge domain, and variants having at least 70% identity thereto.

In an aspect, this application pertains to a use of a peptide dimer or pharmaceutically acceptable salt or solvate thereof in the manufacture of a medicament for treatment of a neurological condition, disease or disorder selected from the group consisting of neural injury, neurological inflammatory or autoimmune disease, and neurodegenerative disease, wherein the peptide dimers comprises a peptide domain independently selected from the leukocyte antigen related (LAR) family phosphatase wedge domains or variants having at least 70% homology thereto.

In an aspect, this application pertains to a process for preparing a peptide dimer or pharmaceutically acceptable salt or solvate thereof, the process comprising combining a first monomer and a second monomer in water and either a) adding an oxidating agent and/or b) oxygenizing the solution, such that the peptide dimer forms, wherein each peptide monomer comprises a peptide domain independently selected from leukocyte antigen related (LAR) family phosphatase wedge domains or variants having at least 65% homologous thereto, a transport moiety, and one cysteine residue or a peptide linker comprising one cysteine residue connecting the peptide domain and the transport moiety, where a disulfide bond is formed between the two cysteine residues on the first and the second peptide monomers.

In yet another aspect, this application pertains to a process for preparing the peptide dimers or pharmaceutically acceptable salt or solvate thereof provided herein, wherein the process comprises combining identical peptide monomers in a solvent with cupric sulfate to allow formation of a non-covalent bond between two peptide monomers, wherein each peptide monomer comprises a peptide domain independently selected from leukocyte antigen related (LAR) family phosphatase wedge domains or variants having at least 70% homology thereto, a transport moiety, and one cysteine residue or a peptide linker comprising one cysteine residue connecting the peptide domain and the transport moiety.

### BRIEF DESCRIPTION OF THE DRAWINGS

The accompanying drawings, which are incorporated herein and constitute part of this specification, illustrate exemplary embodiments of the disclosure, and, together with the general description given above and the detailed description given below, serve to explain the features of the invention.

FIG. 1 is a chromatogram of a purified acetate salt of Compound 8 (TAT-Cys-PTPRS wedge domain dimer) obtained by an oxidative dimerization process where cupric sulfate was used as the oxidative agent.

FIG. 2 graphically depicts the BBB score of the rats receiving Compound 8 as compared to the rats receiving saline at day 7 after SCI, as detailed in Example 3.

FIG. 3 graphically depicts the BBB score of the rats receiving Compound 8 as compared to the rats receiving saline at week 7 after SCI, as detailed in Example 3.

FIG. 4 graphically depicts the BBB score of the rats receiving Compound 8 as compared to rats receiving saline at week 12 after SCI, as detailed in Example 3.

FIG. 5 graphically depicts the weekly average of estimated urine retention in SCI rats treated with Compound 8 in saline and SCI rats treated with a vehicle control (saline) during a course of study.

FIG. 6 graphically depicts the recovery of occasional walking in Compound 8-treated SCI rats, compared to vehicle control treated SCI rats.

FIG. 7 graphically depicts the recovery of frequent walking in Compound 8-treated SCI rats, compared to vehicle control treated SCI rats.

FIG. 8A graphically depicts the effects of Compound 8, compared to Compound 4 and vehicle control, in improving the recovery of improvements in hind limb toe clearance, paw position, trunk stability, and tail position in SCI rats as measured by BBB sub-score. Data are presented as mean±SEM. N=12-13/group.

FIG. 8B graphically depicts the weekly BBB subscores of SCI rats treated with Compound 8 in comparison with animals treated with Compound 4 and saline. Data are presented as distributions of the weekly average BBB subscores. N=12-13/group.

FIG. 9 graphically depicts the percent of SCI rats that achieved BBB sub-score of 1 or better (higher) in the testing group of SCI rats treated with Compound 8, compared to the control group of SCI rats treated with saline alone and the group of SCI rats treated with Compound 4.

FIG. 10 graphically depicts the pharmacokinetic profiles of Compound 7 in rat plasma following intravenous or subcutaneous administration at 1.75 or 10.5 mg/kg, respectively.

FIG. 11 graphically depicts the weekly BBB scores of SCI rats in groups treated with Compound 8 in comparison with animals treated with Compound 4 and saline according to an example of the present disclosure.

FIG. 12 graphically depicts the percentages of SCI rats having BBB scores of 10 or above in groups treated with Compound 8, Compound 4, and saline alone, according to an example of the present disclosure.

FIG. 13 graphically depicts the percentages of SCI rats having BBB scores of 11 in groups treated with Compound 8, Compound 4, and saline alone, according to an example of the present disclosure.

FIG. 14 graphically depicts the average weekly bladder scores of groups of rats treated with Compound 8, Compound 4, and saline alone, according to an example of the present disclosure.

FIG. 15 graphically depicts the percentages of SCI rats with weekly bladder scores of 2 or less (better) in the groups of rats treated with Compound 8, Compound 4, and saline alone, according to an example of the present disclosure.

FIG. 16 graphically depicts the percentage of rats that have reached BBB scores of 10 or above and/or bladder scores of 2 or less at the end of a study disclosed in an example of the present application.

FIG. 17 graphically depicts the percentage of rats that have reached BBB scores of 11 and/or bladder scores of 2 or less at the end of a study according to an example of the present disclosure.

FIG. 18 graphically depicts the stability of Compound 7 in physiological buffer (HBSS), compared with Compound 3, according to an example of the present disclosure.

FIG. 19, FIG. 20, and FIG. 21 graphically depict the stabilities of Compound 7 in rat plasma, dog plasma, and human plasma, compared to Compound 3, according to an example of the present disclosure.

FIG. 22 is a set of transmission electron microscopy images illustrating the distinct patterns of self-assembly of Compound 7 and Compound 3 in water and isotonic saline, according to an example of the present disclosure.

### DETAILED DESCRIPTION

Embodiments of the present disclosure relate to peptide dimers and compositions comprising the peptide dimers for repairing the nervous system of a subject.

Embodiments of the present disclosure further provide pharmaceutical compositions comprising the peptide dimers disclosed herein and methods of using the peptide dimers and/or pharmaceutical compositions disclosed herein for repairing the nervous system of a subject.

In an embodiment, the pharmaceutical compositions provided herein further comprise a pharmaceutically acceptable carrier.

Embodiments of the present disclosure also provide methods of treating diseases, disorders, and/or conditions associated with activation and signaling of the LAR family of phosphatases comprising administering the peptide dimers and/or pharmaceutical compositions disclosed herein to a subject in need thereof.

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless otherwise defined, all scientific and technical terms used herein shall have the meanings that are commonly understood by those of ordinary skill in the art. Further, unless otherwise required by context, singular terms shall include pluralities and plural terms shall include the singular. Generally, nomenclature utilized in connection with, and techniques of, cell and tissue culture, molecular biology, and protein and oligo- or polynucleotide chemistry and hybridization described herein are those well-known and commonly used in the art.

As used herein, “one or more of a, b, and c” means a, b, c, ab, ac, bc, or abc. The use of “or” herein is the inclusive or.

Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, 1%, and ±0.1% from the specified value, as such variations are appropriate to perform the disclosed methods.

### Chemistry

As used herein, the term “alkyl” refers to a straight or branched saturated hydrocarbon. For example, an alkyl group can have 1 to 12 carbon atoms (i.e., (C₁-C₁₂)alkyl), 1 to 6 carbon atoms (i.e., (C₁-C₆)alkyl), 1 to 4 carbon atoms (i.e., (C₁-C₄)alkyl), or 1 to 3 carbon atoms (i.e., (C₁-C₃)alkyl). Examples of alkyl groups include, but are not limited to, methyl (Me, —CH₃), ethyl (Et, —CH₂CH₃), 1-propyl (n-Pr, n-propyl, —CH₂CH₂CH₃), isopropyl (i-Pr, i-propyl, —CH(CH₃)2), 1-butyl (n-bu, n-butyl, —CH₂CH₂CH₂CH₃), 2-butyl (s-bu, s-butyl, —CH(CH₃)CH₂CH₃), tert-butyl (t-bu, t-butyl, —CH(CH₃)₃), 1-pentyl (n-pentyl, —CH₂CH₂CH₂CH₂CH₃), 2-pentyl (—CH(CH₃) CH₂CH₂CH₃), neopentyl (—CH₂C(CH₃)₃), 1-hexyl (—CH₂CH₂CH₂CH₂CH₂CH₃), 2-hexyl (—CH(CH₃)CH₂CH₂CH₂CH₃), heptyl (—(CH₂)₆CH₃), octyl (—(CH₂)₇CH₃), 2,2,4-trimethylpentyl (—CH₂C(CH₃)₂CH₂CH(CH₃)₂), nonyl (—(CH₂)₈CH₃), decyl (—(CH₂)₉CH₃), undecyl (—(CH₂)₁₀CH₃), and dodecyl (—(CH₂)₁₁CH₃). In an embodiment, alkyl refers to C₍₁₋₆₎alkyl. In another embodiment, alkyl refers to C₍₁₋₄₎alkyl. In another embodiment, alkyl refers to C₍₁₋₃₎alkyl.

As used herein, the term “alkylene” refers to a bivalent alkyl group. For example, an alkylene group can have 1 to 12 carbon atoms (i.e., (C₁-C₁₂)alkylene), 1 to 6 carbon atoms (i.e., (C₁-C₆)alkylene), 1 to 2 carbon atoms (i.e., (C₁-C₂)alkylene), or 1 carbon atom (i.e., (C₁)alkylene). Examples of alkylene groups include, but are not limited to, methylene (—CH₂—), ethylene (—CH₂CH₂—), n-propylene (—CH₂CH₂CH₂—), n-butylene (—CH₂CH₂CH₂CH₂—), etc.

### Nucleic Acids

As used herein, the terms “polynucleotide sequence” and “nucleotide sequence” are also used interchangeably herein.

As used herein, the term “wild type” refers to the naturally occurring polynucleotide sequence encoding a protein, or a portion thereof, or protein sequence, or portion thereof, respectively, as it normally exists in vivo. As used herein, the term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “recombinant,” as used herein, means that a protein is derived from a prokaryotic or eukaryotic expression system. As used herein, the term “recombinant” refers to a genetic material formed by a genetic recombination process. A “recombinant protein” is made through genetic engineering. A recombinant protein is coded by a DNA sequence created artificially. A recombinant protein is a protein that is coded by a recombinant nucleic acid sequence. A recombinant nucleic acid sequence has a sequence from two or more sources incorporated into a single molecule.

As used herein, the term “expression cassette” refers to a part of a vector DNA used for cloning and transformation. In each successful transformation, the expression cassette directs the cell's machinery to make polypeptide. Some expression cassettes are designed for modular cloning of protein-encoding sequences so that the same cassette can easily be altered to make different proteins. Expression cassettes may also refer to a recombinantly produced nucleic acid molecule that is capable of expressing a genetic sequence in a cell. An expression cassette typically includes a regulatory region such as a promoter, (allowing transcription initiation), and a sequence encoding one or more proteins or RNAs. Optionally, the expression cassette may include transcriptional enhancers, non-coding sequences, splicing signals, transcription termination signals, and polyadenylation signals. The sequences controlling the expression of the gene, i.e. its transcription and the translation of the transcription product, are commonly referred to as regulatory unit. Most parts of the regulatory unit are located upstream of coding sequence of the heterologous gene and are operably linked thereto. The expression cassette may also contain a downstream 3′ untranslated region comprising a polyadenylation site. The regulatory unit of the invention is either directly linked to the gene to be expressed, i.e. transcription unit, or is separated therefrom by intervening DNA such as for example by the 5′-untranslated region of the heterologous gene. Preferably the expression cassette is flanked by one or more suitable restriction sites in order to enable the insertion of the expression cassette into a vector and/or its excision from a vector. Thus, the expression cassette according to the present invention can be used for the construction of an expression vector, in particular a mammalian expression vector.

As used herein, the term “expression vector,” otherwise known as an expression construct, refers to a plasmid or virus designed for protein expression in cells. The vector is used to introduce a specific gene into a target cell and can commandeer the cell's mechanism for protein synthesis to produce the protein encoded by the gene. The plasmid is engineered to contain regulatory sequences that act as enhancer and promoter regions and lead to efficient transcription of the gene carried on the expression vector. The goal of a well-designed expression vector is the production of significant amount of stable messenger RNA, and therefore proteins.

As used herein, the term “host cell” and the term “host” refer to 1) a cell that harbors foreign molecules, viruses, etc.; 2) a cell that has been introduced with DNA or RNA, such as a bacterial cell acting as a host cell for the DNA isolated from a bacteriophage.

As used herein, a “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a PTPR wedge domain sequence with transport moiety sequence. A fusion protein may be created, for example, by chemical synthesis, or by creating and translating a polynucleotide in which the peptide regions are encoded in the desired relationship. A chimeric protein can further comprise a second amino acid sequence associated with the first amino acid sequence by a covalent, non-peptide bond or a non-covalent bond.

As used herein, the term “modified” and the term “mutant” when made in reference to a gene or to a gene product refer, respectively, to a gene or to a gene product which displays modifications in sequence and/or functional properties (i.e., altered characteristics) when compared to the wild-type gene or gene product.

### Polypeptides

As used herein, the term “amino acid” includes alanine (Ala or A); arginine (Arg or R); asparagine (Asn or N); aspartic acid (Asp or D); cysteine (Cys or C); glutamine (Gin or Q); glutamic acid (Glu or E); glycine (Gly or G); histidine (His or H); isoleucine (lie or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the disclosure and include norleucine, ornithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge but have non-zero partial charges in different portions of their side chains (e.g., M, F, W, S, Y, N, Q, C). These amino acids can participate in hydrophobic interactions and electrostatic interactions. As used herein, the term “charged amino acid” includes amino acids that can have non-zero net charge on their side chains (e.g., R, K, H, E, D). These amino acids can participate in hydrophobic interactions and electrostatic interactions.

As used herein, the terms “peptide” or “polypeptide” are used interchangeably herein and refer to compounds consisting of from about 2 to about 90 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, NY, 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6 aminocaproic acid, sarcosine, statine), and unusual amino acids (e.g., citrulline, homocitrulline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green &amp; Wuts, PROTECTING GROUPS IN ORGANIC SYNTHESIS (John Wiley &amp; Sons, 1991). The functional groups of a peptide can also be derivatized (e.g., alkylated) using art-known methods.

As is clear to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating a “Hy-” moiety at the amino terminus (N-terminus) of the sequence, and either an “—OH” moiety or an “—NH2” moiety at the carboxy terminus (C-terminus) of the sequence. In such cases, and unless otherwise indicated, a “Hy-” moiety at the N-terminus of the sequence in question indicates a hydrogen atom, corresponding to the presence of a free primary or secondary amino group at the N-terminus, while an “—OH” or an “—NH2” moiety at the C-terminus of the sequence indicates a hydroxy group or an amino group, corresponding to the presence of an amido (CONH2) group at the C-terminus, respectively. In each sequence of the invention, a C-terminal “—OH” moiety may be substituted for a C-terminal “—NH2” moiety, and vice-versa.

Peptides can be synthesized and assembled into libraries comprising many discrete molecular species. Such libraries can be prepared using well-known methods of combinatorial chemistry and can be screened as described herein or using other suitable methods to determine if the library comprises peptides of interest. Such peptide can then be isolated by suitable means.

As used herein, the term “peptidomimetic”, refers to a protein-like molecule designed to mimic a peptide. Peptidomimetics typically arise either from modification of an existing peptide, or by designing similar systems that mimic peptides, such as peptoids and @-peptides. Irrespective of the approach, the altered chemical structure is designed to advantageously adjust the molecular properties such as, stability or biological activity. These modifications involve changes to the peptide that do not occur naturally (such as altered backbones and the incorporation of nonnatural amino acids).

As used herein, the term “monomer” or “peptide monomer” refers to a peptide molecule that may bind chemically to other molecules such as another peptide molecule to form a polymer.

As used herein, the term “peptide dimer” refers broadly to a peptide molecule comprising two monomer subunits, which can be identical or different. As such, dimers of the present invention include homodimers and heterodimers.

As used herein, the term “subunit” refers to a separate polypeptide chain that makes a certain protein which is made up of two or more polypeptide chains joined together. In a protein molecule composed of more than one subunit, each subunit can form a stable folded structure by itself. The amino acid sequences of subunits of a protein or polypeptide can be identical, similar, or completely different.

The term “NH₂,” as used herein, can refer to a free amino group present at the amino terminus of a polypeptide. The term “OH,” as used herein, can refer to a free carboxy group present at the carboxy terminus of a peptide. Further, the term “Ac,” as used herein, refers to Acetyl protection through acylation of the C- or N-terminus of a polypeptide. In certain peptides shown herein, the NH2 locates at the C-terminus of the peptide indicates an amino group.

As used herein, the term “linker” and the term “peptide linker” are interchangeable and refer to short peptide sequences that occur between functional protein domains and link the functional domains together. Linkers designed by researchers are generally classified into three categories according to their structures: flexible linkers, rigid linkers, and in vivo cleavable linkers. A flexible linker is often composed of flexible residues like glycine and serine so that the adjacent protein domains are free to move relative to one another. A linker also may play a role in releasing the free functional domain in vivo (as in in vivo cleavable linkers). Linkers may offer many other advantages for the production of fusion proteins, such as improving biological activity, increasing expression yield, and achieving desirable pharmacokinetic profiles. The composition and length of a linker may be determined in accordance with methods well known in the art and may be tested for efficacy. A linker may be from about 3 to about 15 amino acids long. In some embodiments of the present invention, a linker may be about 5 to about 10 amino acids long, however, longer linker may be used in embodiments of the present invention.

As used herein, the terms “portion”, “fragment”, “variant”, “derivative” and “analog”, when referring to a polypeptide of the present invention include any polypeptide that retains at least some biological activity referred to herein (e.g., inhibition of an interaction such as binding). Polypeptides as described herein may include portion, fragment, variant, or derivative molecules without limitation, as long as the polypeptide still serves its function. Polypeptides or portions thereof of the present invention may include proteolytic fragments, deletion fragments and in particular, or fragments that more easily reach the site of action when delivered to an animal.

As used herein, the term “protein purification” refers to a series of processes intended to isolate one or a few proteins or polypeptides from a complex mixture, such as cell culture media, cells, tissues or whole organisms, etc. Usually, a protein purification protocol contains one or more chromatographic steps. The basic procedure in chromatography is to flow the solution containing the protein through a column packed with various materials. Different proteins interact differently with the column material and can thus be separated by the time required to pass the column, or the conditions required to elute the protein from the column. Many purification strategies exist. For example, a protein can be attached with an antigen peptide tag by engineering and be purified using an antibody against the antigen peptide tag. Usually, during purification, the protein with an antigen peptide tag can be added on a column loaded with resin that is coated with an antibody or by incubating with a loose resin that is coated with an immobilizing antibody. This particular procedure is known as immunoprecipitation. Immunoprecipitation is quite capable of generating an extremely specific interaction which usually results in binding only the desired protein. The purified tagged proteins can then easily be separated from the other proteins in solution and later eluted back into clean solution.

In some embodiments, the dimers disclosed herein are substantially isolated. By “substantially isolated” it is meant that the dimer is at least partially or substantially separated from the environment in which it was formed or detected. Partial separation can include, for example, a dimer enriched in the compound of the invention. Substantial separation can include compositions containing at least about 50%, at least about 60%, at least about 70%, at least about 80%, at least about 90%, at least about 95%, at least about 97%, or at least about 99% by weight of the dimer.

As used herein, the term “inhibitor” refers to a molecule, compound, or agent that reduces or inhibits at least one activity, signaling, or function of leukocyte-common antigen related (LAR) family of phosphatases induced by proteoglycan, reduces in the activity, signaling, and/or function of chondroitin sulfate proteoglycan (CSPG), and/or the interaction between chondroitin sulfate proteoglycan (CSPG) and LAR family of phosphatases. In some embodiments, “inhibitor” also refers to a molecule, compound, or agent that abolish inhibitory effects of CSPGs on neural cells activated with CSPGs. In various embodiments, inhibitors disclosed herein are peptide dimers comprising an amino acid sequence derived from a cytoplasmic wedge domain of a receptor-type protein-tyrosine phosphatase (PTPR).

### Homology

As used herein, the terms “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

As used herein, the term “analogue” and the term “analog” refer to one of a group of chemical compounds that share structural and/or functional similarities but are different in respect to elemental composition. A structural analog is a compound having a structure similar to that of another one, but differing from it in respect of one or more components, such as one or more atoms, functional groups, or substructures, etc. Functional analogs are compounds that have similar physical, chemical, biochemical, or pharmacological properties. Functional analogs are not necessarily also structural analogs with a similar chemical structure.

As used herein, the term “sequence identity,” “percent identity,” “percent homology,” or, for example, comprising a “sequence 80% identical to,” refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using an NWSgapdna.CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

For instance, the peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

### Compositions and Formulations

As used herein, the term “pharmaceutically acceptable” refers to a compound or drug approved or approvable by a regulatory agency of a federal or a state government, listed or listable in the U.S. Pharmacopeia or in other generally recognized pharmacopeia for use in mammals, including humans.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the disclosure with a pharmaceutically acceptable carrier. The pharmaceutical composition facilitates administration of the compound to a subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the subject. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein, the term “pharmaceutical formulation” and the term “drug formulation” refer to a mixture or a structure in which different chemical substances, including the active drug, are combined to form a final medicinal product, such as a sterile product, a solution, a powder, an emulsion, a capsule, a tablet, a granule, a topical preparation, a non-conventional product such as semi-solid or sustained-release preparations, liquid, etc. Pharmaceutical formulation is prepared according to a specific procedure, a “formula.” The drug formed varies by the route of administration.

### Medical Intervention

As used herein, the term “dose” refers to a specified amount of medication taken at one time. A “daily dose” refers to the total dosage amount administered to an individual in a single 24-hour day.

As used herein, the term “mg/kg” refers to the dose of a substance administered to an individual in milligrams per kilogram of body weight of the individual.

As used herein, the term “dosage” refers to the administering of a specific amount, number, and frequency of doses over a specified period of time. Dosage implies duration. A “dosage regimen” is a treatment plan for administering a drug over a period of time.

As used herein, the phrases “parenteral administration” and “administered parenterally” as used herein means modes of administration other than enteral and topical administration or through the digestive tract, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intracisternal injection and infusion.

As used herein, the phrases “systemic administration,” “administered systemically,” “peripheral administration” and “administered peripherally” as used herein mean the administration of a compound, drug or other material other than directly into a target tissue (e.g., the nervous system), such that it enters the animal's system and, thus, is subject to metabolism and other like processes, for example, subcutaneous administration.

As used herein, the term “patient” or “subject” or “animal” or “host” refers to any mammal. The subject may be a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

As used herein, the term “administering” to a patient includes dispensing, delivering or applying an active compound in a pharmaceutical formulation to a subject by any suitable route for delivery of the active compound to the desired location in the subject (e.g., to thereby contact a desired cell such as a desired neuron), including administration into the cerebrospinal fluid or across the blood-brain barrier, delivery by either the parenteral or oral route, intramuscular injection, subcutaneous or intradermal injection, intravenous injection, buccal administration, transdermal delivery and administration by the rectal, colonic, vaginal, intranasal or respiratory tract route. The agents may, for example, be administered to a comatose, anesthetized, or paralyzed subject via an intravenous injection or may be administered intravenously to a pregnant subject to stimulate axonal growth in a fetus. Specific routes of administration may include topical application (such as by eyedrops, creams or erodible formulations to be placed under the eyelid, intraocular injection into the aqueous or the vitreous humor, injection into the external layers of the eye, such as via subconjunctival injection or subtenon injection, parenteral administration or via oral routes.

The term “treat,” “treated,” “treating,” or “treatment” includes the diminishment or alleviation of at least one symptom associated or caused by the state, disorder or disease being treated. The term “treatment” as used herein also includes: (1) inhibiting the disease or condition, i.e., arresting the development or progression of the disease or condition, (2) relieving the disease or condition, i.e., causing the condition to regress, (3) stopping the symptoms of the disease, and/or (4) enhancing the conditions desired.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, an “effective amount,” of an agent or therapeutic peptide dimers disclosed herein is an amount sufficient to achieve a desired therapeutic or pharmacological effect, such as an amount that is capable of activating the growth of neurons. An effective amount of an agent as defined herein may vary according to factors such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects. As used herein, the term a “therapeutically effective amount” refers to an amount effective, at dosages and for periods of time necessary, to achieve the desired therapeutic result. A therapeutic result may be, e.g., lessening of symptoms, prolonged survival, improved mobility, and the like. A therapeutic result need not be a “cure.”

As used herein, the terms “improve,” “improving” or “improvement” or grammatical variations thereof used in relation to behaviors refer to the ability to achieve a measurable increase in performance in relation to tasks used to test these behaviors in a subject, including humans or non-human animals.

### Central Nervous System

As used herein the term, “central nervous system (CNS) neurons” include the neurons of the brain, the cranial nerves, and the spinal cord.

As used herein, the term “peripheral nervous system (PNS) neurons” includes the neurons which reside or extend outside of the CNS. PNS is intended to include the neurons commonly understood as categorized in the peripheral nervous system, including sensory neurons and motor neurons.

As used herein, the term “contacting neurons” or “treating neurons” refers to any mode of agent delivery or “administration,” either to cells or to whole organisms, in which the agent is capable of exhibiting its pharmacological effect in neurons. “Contacting neurons” includes both in vivo and in vitro methods of bringing an agent of the invention into proximity with a neuron. Suitable modes of administration can be determined by those skilled in the art and such modes of administration may vary between agents. For example, when axonal growth of neurons is stimulated ex vivo, agents can be administered, for example, by transfection, lipofection, electroporation, viral vector infection, or by addition to growth medium.

As used herein, the term “neurological disorder” includes a disease, disorder, or condition which directly or indirectly affects the normal functioning or anatomy of a subject's nervous system. The term “stroke” is art-recognized and includes sudden diminution or loss of consciousness, sensation and voluntary motion caused by rupture or obstruction (for example, by a blood clot) of an artery of the brain. “Traumatic brain injury” is art-recognized and includes the condition in which a traumatic blow to the head causes damage to the brain or connecting spinal cord, with or without penetrating the skull. Usually, the initial trauma can result in expanding hematoma, subarachnoid hemorrhage, cerebral edema, raised intracranial pressure, and cerebral hypoxia, which can, in turn, lead to severe secondary events due to low cerebral blood flow.

As used herein, the term axonal “growth” or “outgrowth” (also referred to herein as “neuronal outgrowth”) includes the process by which axons or dendrites extend from a neuron. The outgrowth can result in a new neuritic projection or in the extension of a previously existing cellular process. Axonal outgrowth may include linear extension of an axonal process by five cell-diameters or more. Neuronal growth processes, including neuritogenesis, can be evidenced by GAP-43 expression detected by methods such as immunostaining. “Stimulating axonal growth” means promoting axonal outgrowth.

As used herein, the term “dieback” refers to axonal retraction that occurs as a result of trauma to the axon.

As used herein, the term “retraction” refers to the receding of the axon away from the site of injury, such as from where the glial scar forms. Here, the end of regenerating axons stops extending and become dystrophic. These dystrophic ends then can recede further from the glial scar and the site of injury.

As used herein, the term “neuronal migration” refers to the ability of neuronal cells to migrate or neuronal processes to migrate such as an axonal or dendritic migration.

### Peptide Dimers

This application relates to compositions and methods for repairing the nervous system in a subject in need thereof. This application also relates to methods and compositions of treating diseases, disorders, and/or conditions associated with the function of LAR family phosphatases.

In an aspect, provided herein is a pharmaceutical composition comprising a peptide that is a dimer comprising a first monomer covalently cross-linked with a second monomer, each monomer comprising a domain comprising an amino acid sequence derived from a cytoplasmic wedge domain of a receptor-type protein-tyrosine phosphatase (PTPR), and wherein the mass ratio of the dimer to free monomer in the pharmaceutical composition is greater than 1:20.

In an embodiment, the pharmaceutical compositions provided herein further comprise a dimer comprising a transport moiety attached to the domain via one cysteine residue or a peptide linker comprising one cysteine residue.

In an embodiment, the pharmaceutical compositions provided herein further comprise a dimer, wherein the dimer improves neural cell repair.

In yet another embodiment, the pharmaceutical compositions provided herein further comprise the first monomer and the second monomer each independently comprising a first domain comprising an amino acid sequence that is at least about 65% identical, at least about 70% identical, at least about 75% identical, at least about 80% identical, at least about 85% identical, at least about 90% identical, at least about 95% identical, or at least about 97% identical to the amino acid sequence of SEQ ID NO:5, 6, or 7; a second domain comprising an amino acid sequence that is at least about 65% identical, at least about 70% identical, at least about 75% identical, at least about 80% identical, at least about 85% identical, at least about 90% identical, at least about 95% identical, or at least about 97% identical to the amino acid sequence of SEQ ID NO:8, 9, 10, or 11; and a cysteine residue; wherein the dimer comprises a chemical linker or bond between the cysteine residue of the first monomer and the cysteine residue of the second monomer.

In an embodiment, the pharmaceutical compositions provided herein further comprise the first monomer and the second monomer each independently comprising an amino acid sequence that is at least 70% identical to the amino acid sequence of SEQ ID NOS:1, 2, 3, or 4. In another embodiment, the pharmaceutical compositions provided herein further comprise the first monomer and the second monomer each independently comprising an amino acid sequence that is identical to the amino acid sequence of SEQ ID NOS:1, 2, 3, or 4. In yet another embodiment, the pharmaceutical compositions provided herein further comprise the dimer comprising identical monomers. In still another embodiment, the pharmaceutical compositions provided herein further comprise the dimer comprising non-identical monomers and the ratio is calculated based on combined total of free monomer. In another embodiment, the pharmaceutical compositions provided herein further comprise the first monomer and the second monomer of the dimer having different C-terminal modifications.

In yet another aspect, provided herein is a pharmaceutical agent for repairing the nervous system of a subject comprising a peptide dimer or pharmaceutically acceptable salt or solvate thereof, comprising two subunits, wherein each subunit comprises a peptide domain independently selected from receptor-type protein-tyrosine phosphatase (PTPR) wedge domains or variants having at least 70% homology thereto. In an embodiment, a pharmaceutical agent provided herein further comprises a peptide dimer or pharmaceutically acceptable salt or solvate thereof, comprising two subunits, wherein each subunit comprises a transport moiety attached to the peptide domain via one cysteine residue or a peptide linker comprising one cysteine residue, wherein the two subunits are covalently cross-linked via a chemical linker between the cysteine residues on each subunit. In another embodiment, a pharmaceutical agent provided herein further comprises a chemical linker chosen from a disulfide bond, a thioether bond, or a thioester bond In still another embodiment, a pharmaceutical agent provided herein further comprises an amino acid sequence having at least 70% identity to the amino acid sequence of SEQ ID NO:8, 9, 10, or 11.

In another embodiment, a pharmaceutical agent provided herein further comprises a transport moiety comprising an amino acid sequence having at least 65% identity to wild type HIV TAT. In yet another embodiment, a pharmaceutical agent provided herein further comprises a TAT sequence comprising an amino acid sequence that is at least 65% identical to the amino acid sequence of SEQ ID NO:5, 6, or 7. In still another embodiment, a pharmaceutical agent provided herein further comprises an amino acid sequence that is at least 70% identical to the amino acid sequence of SEQ ID NOS:1, 2, 3, or 4.

In an embodiment, a pharmaceutical agent provided herein further comprises subunits comprising different C-terminal modifications. In another embodiment, a pharmaceutical agent provided herein further comprises a peptide dimer comprises two different subunits. In yet another embodiment, a pharmaceutical agent provided herein further comprises the peptide dimer comprising two identical monomer subunits. In still another embodiment, a pharmaceutical agent provided herein further comprises a peptide dimer or pharmaceutically acceptable salt or solvate thereof has a purity of at least 90%.

In an embodiment, a pharmaceutical composition provided herein further comprises the pharmaceutical agent provided herein. In another embodiment, a pharmaceutical composition does not comprise DMSO. In still another aspect, provided herein is a use of the pharmaceutical agent provided herein in the manufacture of a medicament for repairing the nervous system and/or treatment of a neurological condition, disease or disorder selected from the group consisting of neural injury, neurological disease caused by inflammation or autoimmunity, and neurodegenerative disease.

In another aspect, provided herein is a method of repairing the nervous system and/or treating a neurological condition, disease or disorder selected from the group consisting of neural injury, neurological disease caused by inflammation or autoimmunity, and neurodegenerative disease, in a subject in need thereof, the method comprising administering an effective amount of the pharmaceutical agent provided herein to a subject in need thereof.

In some embodiments, the ratio of dimer to free monomer in the pharmaceutical composition is greater than 1:19, greater than 1:18, greater than 1:17, greater than 1:16, greater than 1:15, greater than 1:14, greater than 1:13, greater than 1:12, greater than 1:11, greater than 1:10, greater than 1:9, greater than 1:8, greater than 1:7, greater than 1:6, greater than 1:5, greater than 1:4, greater than 1:3, greater than 1:2, or greater than 1:1. In some embodiments, the ratio of dimer to free monomer in the pharmaceutical composition is greater than 1:1.

In some embodiments, the ratio of dimer to free monomer in the pharmaceutical composition is greater than 2:1, greater than 3:1, greater than 4:1, greater than 5:1, greater than 6:1, greater than 7:1, greater than 8:1, greater than 9:1, greater than 10:1, greater than 11:1, greater than 12:1, greater than 13:1, greater than 14:1, greater than 15:1, greater than 16:1, greater than 17:1, greater than 18:1, greater than 19:1, or greater than 20:1. In some embodiments, the ratio of dimer to free monomer in the pharmaceutical composition is greater than 4:1. In some embodiments, the ratio of dimer to free monomer in the pharmaceutical composition is up to 10:1, up to 15:1, up to 20:1, up to 25:1, or up to 27:1. In some embodiments, the ratio of dimer to free monomer in the pharmaceutical composition is greater than 10:1 (up to 27:1).

In still another embodiment, the ratio is calculated based on combined total of free monomer.

The first and second monomer of the dimer may be connected via any method known in the art. In some embodiments, the first and second monomer are connected via a bond. In some embodiments, the first and second monomer are connected via a cystine bridge. In some embodiments, the dimer comprises a linker between the first monomer and the second monomer. In some embodiments, the dimer comprises a disulfide linkage between the first monomer and the second monomer.

In some embodiments, the dimer comprises a transport moiety that facilitates uptake of the dimer by the cell. In some embodiments, the transport moiety may be an HIV TAT transport moiety (i.e., a TAT sequence). Transport moieties can be repeated more than once in the dimer. The repetition of a transport moiety may affect (e.g., increase) the uptake of the dimer by a desired cell. In either or both of the first and second monomer, the transport moiety may be located either at the amino-terminal region or the carboxy-terminal region or at both regions. In one embodiment, the transport moiety is located at the N-terminal region of the monomers.

In some embodiments, the transport moiety is connected via a peptide linker. In some embodiments, the transport moiety is connected via two peptide linkers.

In some embodiments, the transport moiety can include at least one transport peptide sequence that allows the dimer to penetrate into the cell by a receptor-independent mechanism. In some embodiment, the dimer is a synthetic peptide that contains a TAT-mediated protein delivery sequence.

Other examples of known transport moieties, subdomains and the like are described in, for example, Canadian patent application No. 2,301,157 (conjugates containing homeodomain of antennapedia), PCT international publication number WO 99/11809, as well as in U.S. Pat. Nos. 5,652,122, 5,670,617, 5,674,980, 5,747,641, 5,804,604, and Bruno P. Meloni, et. Al. Frontiers in Neurology. 2020; 11 (Article 108): 1-28, all of which are incorporated herein by reference in their entirety. Thus, in some embodiments, the transport moiety is an HIV TAT peptide; a herpes simplex virus-1 DNA binding protein VP22 peptide, an amino acid region of the third alpha-helix of antennapedia homeodomain, a Histidine tag ranging in length from 4 to 30 histidine repeats, a variation derivative or homologue thereof capable of facilitating uptake of the active cargo moiety by a receptor independent process, or a cationic arginine-rich peptide (CARP). In some embodiments, the transport moiety may be chosen from neuroprotective CARPs that possess the following properties: (i) range in size from 4 to 40 amino acids; (ii) positive net charge≥+2 to +20; (iii) one or more positively charged arginine residues that comprise between 20 and 100% of the peptide; (iv) other positively charged amino acids namely lysine and histidine; (v) amphiphilicity due to the presence of both hydrophilic (e.g., arginine, lysine) and hydrophobic (e.g., tryptophan, phenylalanine, tyrosine) amino acids; and (vi) endocytic and/or non-endocytic cell membrane traversing properties, including the ability to cross the blood-brain and blood-spinal cord barriers (BBB/BSCB). In certain embodiments, the transport moiety may be a cationic arginine-rich peptide fused to TAT.

In addition, the transport moiety(ies) can include polypeptides having a basic amino acid rich region. As used herein, the term “basic amino acid rich region” relates to a region of a protein or peptide with a high content of the basic amino acids such as arginine, histidine, asparagine, glutamine, lysine. A “basic amino acid rich region” may have, for example 15% or more of basic amino acid. In some instance, a “basic amino acid rich region” may have less than 15% of basic amino acids and still function as a transport agent region. In other instances, a basic amino acid region will have 30% or more of basic amino acids.

The transport moiety(ies) may further include a proline rich region. A proline rich region refers to a region of a polypeptide containing more prolines than what is generally observed in naturally occurring proteins (e.g., proteins encoded by the human genome). As used herein, the term proline rich region refers to a region of a polypeptide with 5% or more (up to 100%) of proline in its sequence. In some instances, a proline rich region may have between 5% and 15% of prolines. Proline rich regions of this application can function as a transport agent region.

Accordingly, in some embodiments, the dimer comprises a transport moiety and a cysteine residue. In some embodiments, the dimer comprises a transport moiety comprising an amino acid sequence having at least 65%, at least 70%, at least 75%, at least 80%, at least 85%, at least 90%, or at least 95% identity to wild type HIV TAT sequence and a cysteine residue.

In some embodiments, the transport moiety comprises an amino acid sequence that is at least 65% identical, at least 70% identical, at least 75% identical, at least 80% identical, at least 85% identical, at least 90% identical, at least 95% identical, or 100% identical to the amino acid sequence of SEQ ID NO:5, 6, or 7 as shown in Table 1 below. In one embodiment, the transport moiety included in the first monomer unit and the second monomer are identical to the amino acid sequence of SEQ ID NO:5.

TABLE 1
TAT Sequences

| Sequence | SEQ ID NO |
|---|---|
| GRKKRRQRRR | 5 |
| YGRKKRRQRRR | 6 |
| GRKKRRQRRRPQ | 7 |

In some embodiments, the dimer comprises a domain selected from the group consisting of a PTPRF wedge domain, a PTPRD wedge domain, and a PTPRS wedge domain, and variants having at least 70% identity thereto. In some embodiments, the dimer comprises a domain selected from the group consisting of a PTPRF wedge domain, a PTPRD wedge domain, and a PTPRS wedge domain, and variants having at least about 65% identity thereto, at least about 70% identity thereto, at least about 75% identity thereto, at least about 80% identity thereto, at least about 85% identity thereto, at least about 90% identity thereto, at least about 95% identity thereto, or at least about 97% identity thereto. In some embodiments, the domain comprises an amino acid sequence that is at least 70% identical to the amino acid sequence of SEQ ID NO:8, 9, 10, or 11 as listed in Table 2. In some embodiments, the domain comprises an amino acid sequence that is at least about 65% identical, at least about 70% identical, at least about 75% identical, at least about 80% identical, at least about 85% identical, at least about 90% identical, at least about 95% identical, or at least about 97% identical to the amino acid sequence of SEQ ID NO:8, 9, 10, or 11 as listed in Table 2.

TABLE 2
Wedge domain sequence of LAR family phosphatases

| Parent PTPR | Wedge Domain Sequence | SEQ ID NO |
|---|---|---|
| PTPRF | DLADNIERLKANDGLKFSQEYESI | 8 |
| PTPRD | ELADHIERLKANDNLKFSQEYESI | 9 |
| PTPRS (human) | DMAEHTERLKANDSLKLSQEYESI | 10 |
| PTPRS (rodent) | DMAEHMERLKANDSLKLSQEYESI | 11 |

In some embodiments, the first monomer and the second monomer each independently comprise a first domain comprising an amino acid sequence that is at least 70% identical to the amino acid sequence of SEQ ID NO:5, 6 or 7; a second domain comprising an amino acid sequence that is at least 70% identical to the amino acid sequence of SEQ ID NO:8, 9, 10, or 11; and a cysteine residue; wherein the dimer comprises a disulfide linkage between the cysteine residue of the first monomer and the cysteine residue of the second monomer. In one embodiment, the first domain comprises an amino acid sequence that is identical to the amino acid sequence of SEQ ID NO:5, 6, or 7. In one embodiment, the transport moiety included in the first monomer unit and the second monomer are identical to the amino acid sequence of SEQ ID NO:5. In one embodiment, the second domain comprises an amino acid sequence that is identical to the amino acid sequence of SEQ ID NO:8, 9, 10, or 11.

In some embodiments, the peptide dimer disclosed herein has the following structure:

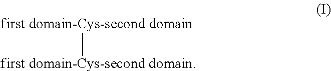

In some embodiments, the “first domain” corresponds to a transport moiety described above and the “second domain” corresponds to a peptide domain as described above.

In some embodiments, the peptide dimer disclosed herein has the following structure:

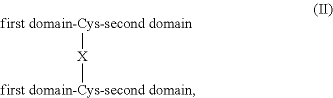

wherein X is a bond or a chemical linker between the two cysteine residues.

In some embodiments, X is a bond, e.g., a disulfide bond between the thiol groups of the two cysteine residues. In some embodiments, X is a chemical linker between the two cysteine residues. The chemical linker may, for example, comprise covalent bonds with each of the thiol groups of the cysteine residues (e.g., disulfide, thioether, or thioester bonds). In some embodiments, the chemical linker is between 5 Å and 50 Å in length. In some embodiments, the chemical linker is between 5 Å and 35 Å in length. In some embodiments, the chemical linker is between 10 Å and 25 Å in length. In some embodiments, the chemical linker consists of atoms selected from C, N, S, O, and H. In some embodiments, the chemical linker comprises atoms selected from C, N, S, O, and H. In some embodiments, the chemical linker consists of atoms selected from C, O, and H. In some embodiments, the chemical linker comprises atoms selected from C, O, and H. In some embodiments, the chemical linker comprises between 1 and 8 carbon atoms. In some embodiments, the chemical linker comprises an alkylene chain (e.g., a C₍₁₋₂₎alkylene, a C₍₁₋₆₎alkylene, or a C₍₁₋₃₎alkylene). In some embodiments, the chemical linker comprises an alkylene chain wherein one or more carbon atoms is replaced with oxygen (e.g., a bivalent polyethylene glycol chain).

In some embodiments, the first monomer and the second monomer each independently comprise an amino acid sequence that is at least 65% identical, at least 70% identical, at least 75% identical, at least 80% identical, at least 85% identical, at least 90% identical, or at least 95% identical to the amino acid sequence of SEQ ID NOS:1, 2, 3, or 4. In an embodiment, the first monomer and the second monomer each independently comprise an amino acid sequence that is identical to the amino acid sequence of SEQ ID NOS:1, 2, 3, or 4.

In an embodiment, the dimer comprises identical monomers. In another embodiment, the dimer comprises non-identical monomers.

In an embodiment, the first monomer and the second monomer of the dimer have different C-terminal modifications.

One potential mechanism for regulation, modulation, and/or inhibition of LAR family of phosphatases involves dimerization of the intracellular portion of the phosphatase. In contrast to receptor tyrosine kinases, which are active as dimers and inactive as monomers, several protein tyrosine phosphatases (PTPs) have been shown to be inactive as dimers and active as monomers. PTPalpha, PTP1B and CD45, which have been crystalized in both forms, have been shown to be active as monomers and inactive as dimers. As PTPRF demonstrates homophillic binding under specific oxidative conditions, and PTPRS can dimerize in response to ligand binding, it is suggested that ligands to LAR family phosphatases can direct the activation state of PTPRF and PTPRS. Therefore, mimicking dimerization with intracellular-targeted therapies may directly inactivate LAR family of phosphatases without alteration of the extracellular matrix or other ligands.

Peptide mimetics of the intracellular portion of the LAR family of phosphatase, when delivered into a neural cell, may inhibit and/or reduce LAR activity or function induced by CSPG. Suppression of LAR family activity, signaling, and/or function in response to CSPG activation was found to promote neural cell outgrowth, including restoration of growth cone motility, extension of processes, sprouting, promotion of neural cell survival and plasticity, and inhibit neural cell dieback.

In some embodiments, the function of a LAR family phosphatase is inhibited or reduced by a peptide or small molecule therapeutic agent that binds to and/or complexes with the intracellular domain of at least one LAR family phosphatase. In some embodiments, one or more of the activities and signaling of the LAR family phosphatase is inhibited or reduced by a peptide or small molecule therapeutic agent that binds to and/or complexes with the intracellular domain of at least one LAR family phosphatase. Accordingly, therapeutic peptides or small molecules that bind to and/or complex with the intracellular domain of at least one LAR family phosphatase of neural cells can be used to promote cell growth, motility, survival and plasticity of these cells.

In some embodiments, the therapeutic agent is a peptide mimetic of the wedge-shaped domain (i.e., wedge domain) of a LAR family phosphatase. Structural and sequence analysis has revealed that all members of the LAR family contain a conserved 24 amino acid wedge-shaped helix-loop-helix motif in the first intracellular catalytic domain that can potentially mediate homo/heterophilic receptor interaction.

Table 3 lists the amino acid sequences of intracellular portions of the LAR family phosphatase members that contain the wedge domain. The 24 amino acid wedge domains of these intracellular portions of LAR family phosphatases are identified by underlining. While the specific structure of the wedge domain is conserved through most LAR family wedge domains, the exact amino acids that make up the wedge domains vary between individual proteins and sub-families.

As can be seen in Table 3, the wedge domain is highly conserved across members of the LAR family. For example, the wedge domain sequence of PTPRS is highly conserved among mammals, with only a single amino acid change in mice and rats (Threonine to Methionine at position 6).

TABLE 3
Wedge Domain of LAR Family Phosphatase
PTPRF Wedge Domain Alignment

| | | | | |
|---|---|---|---|---|
| Mouse | 1338 | PIPITDLADNIERLICANDGICLFSQEYESIDPGQ | 1370 | SEQ ID NO: 12 |
| Rat | 1338 | PIPITDLADNIERLICANDGKLFSQEYESIDPGQ | 1370 | SEQ ID NO: 13 |
| Human | 1347 | PIPITDLADNIERLICANDGKLFSQEYESIDPGQ | 1379 | SEQ ID NO: 14 |

PTPRS Wedge Domain Alignment

| | | | | |
|---|---|---|---|---|
| Mouse | 1347 | PIPITDMAEHMERLKANDSLKLSOEYESIDPGQ | 1379 | SEQ ID NO: 15 |
| Rat | 1303 | PIPITDMAEHMERLKANDSLKLSQEYESIDPGQ | 1335 | SEQ ID NO: 16 |
| Human | 1368 | PIPIADMAEHTERLKANDSLKLSOEYESIDPGQ | 1400 | SEQ ID NO: 17 |

PTPRD Wedge Domain Alignment

| | | | | |
|---|---|---|---|---|
| Mouse | 1326 | PIPILELADHIERLKANDNLKFSQEYESIDPGQ | 1379 | SEQ ID NO: 18 |
| Human | 1335 | PIPILELADHIERLKANDNLICFSOEYESIDPGQ | 1367 | SEQ ID NO: 19 |

Preferred embodiments are listed in Tables 4, 5, and 6. Provided herein are monomer compounds 1, 2, 3, and 4, as well as methods of treating a subject suffering from any of the indications provided herein by administering to the subject compounds 1, 2, 3, or 4.

TABLE 4
Monomers

| Cmpd | | Sequence | |
|---|---|---|---|
| 1 | TAT-Cys- | GRKKRRQRRRCDLADNIER | SEQ ID |
| | PTPRF | LKANDGLKFSQEYESI | NO: 1 |
| 2 | TAT-Cys- | GRKKRRQRRRCELADHIER | SEQ ID |
| | PTPRD | LKANDNLKFSQEYESI | NO: 2 |
| 3 | TAT-Cys- | GRKKRRQRRRCDMAEHTER | SEQ ID |
| | PTPRS | LKANDSLKLSQEYESI | NO: 3 |
| 4 | TAT-Cys- | GRKKRRQRRRCDMAEHMER | SEQ ID |
| | rodent PTPRS | LKANDSLKLSQEYESI | NO: 4 |

TABLE 5
Homodimers

| | | Amino acid sequence | |
|---|---|---|---|
| Cmpd | Product | indicating disulfide linkage | |
| 5 | TAT-Cys- | GRKKRRQRRRCDLADNIERLKANDGLKFSQEYESI | SEQ ID |
| | PTPRF | \| | NO: 1 |
| | wedge | GRKKRRQRRRCDLADNIERLKANDGLKFSQEYESI | SEQ ID |
| | domain | | NO: 1 |
| | homodimer | | |
| 6 | TAT-Cys- | GRKKRRQRRRCELADHIERLKANDNLKFSQEYESI | SEQ ID |
| | PTPRD | \| | NO: 2 |
| | wedge | GRKKRRQRRRCELADHIERLKANDNLKFSQEYESI | SEQ ID |
| | domain | | NO: 2 |
| | homodimer | | |
| 7 | TAT-Cys- | GRKKRRQRRRCDMAEHTERLKANDSLKLSQEYESI | SEQ ID |
| | human | \| | NO: 3 |
| | PTPRS | GRKKRRQRRRCDMAEHTERLKANDSLKLSQEYESI | SEQ ID |
| | wedge | | NO: 3 |
| | domain | | |
| | homodimer | | |
| 8 | TAT-Cys- | GRKKRRQRRRCDMAEHMERLKANDSLKLSQEYESI | SEQ ID |
| | rodent | \| | NO: 4 |
| | PTPRS | GRKKRRQRRRCDMAEHMERLKANDSLKLSQEYESI | SEQ ID |
| | wedge | | NO: 4 |
| | domain | | |
| | homodimer | | |

“|” represents Cys-Cys disulfide.

TABLE 6
Heterodimers

| | Heterodimer | Amino acid sequence | |
|---|---|---|---|
| Cmpd | Product | indicating disulfide linkage | |
| 9 | TAT-Cys- | GRKKRRQRRRCDLADNIERLKANDGLKFSQEYESI | SEQ ID |
| | PTPRF | \| | NO: 1 |
| | TAT-Cys- | GRKKRRQRRRCELADHIERLKANDNLKFSQEYESI | SEQ ID |
| | PTPRD | | NO: 2 |
| 10 | TAT-Cys- | GRKKRRQRRRCELADHIERLKANDNLKFSQEYESI | SEQ ID |
| | PTPRD | \| | NO: 2 |
| | TAT-Cys- | GRKKRRQRRRCDMAEHTERLKANDSLKLSQEYESI | SEQ ID |
| | PTPRS | | NO: 3 |
| 11 | TAT-Cys- | GRKKRRQRRRCDMAEHTERLKANDSLKLSQEYESI | SEQ ID |
| | PTPRS | \| | NO: 3 |
| | TAT-Cys- | GRKKRRQRRRCDLADNIERLKANDGLKFSQEYESI | SEQ ID |
| | PTPRF | | NO: 1 |

“|” represents Cys-Cys disulfide.

In some embodiments, the amino acid residue 17T in SEQ ID NO:3 may respectively be substituted with M, resulting in rat and mouse PTPRS variant.

In some embodiments, the peptide dimer disclosed herein may comprise a peptide domain that is at least 65%, at least 70%, at least 75%, at least 80%, at least 85%, at least 90%, at least 95%, or about 100% identical to about 10 consecutive amino acids of the wedge domain of a LAR family phosphatase. In some embodiments, the peptide dimer disclosed herein may comprise a peptide domain that is at least 65%, at least 70%, at least 75%, at least 80%, at least 85%, at least 90%, at least 95%, or about 100% identical to about 15 consecutive amino acids of the wedge domain of a LAR family phosphatase. In some embodiments, the peptide dimer disclosed herein may comprise a peptide domain that is at least 65%, at least 70%, at least 75%, at least 80%, at least 85%, at least 90%, at least 95%, or about 100% identical to about 20 consecutive amino acids of the wedge domain of a LAR family phosphatase. The peptide dimer can modulate the signaling and/or function of a LAR family phosphatase in cells expressing the LAR family phosphatase, such as neural cells.

In some embodiments, the peptide dimer comprises a peptide domain that is at least 65%, at least 70%, at least 75%, at least 80%, at least 85%, at least 90%, at least 95%, or about 100% identical to an about 10 to about 20 amino acids consecutive amino acids of a wedge domain of PTPRS, and the peptide dimer can promote cell viability, morphogenesis or differentiation by subject to inhibitory effects of CSPGs on cells, e.g., neural cells.

In some embodiments, the peptide dimer disclosed herein comprises a peptide linker. In some embodiments, the peptide linker is in the middle of a monomer subunit connecting the transport moiety and the first or the second peptide domain. In some embodiment, each peptide monomer includes a peptide linker having one cysteine residue such that the first monomer subunit and the second monomer subunit are cross-linked via the disulfate bond between the two cysteine residues in the first and the second monomer subunits.

In some embodiments, the monomer subunits of the peptide dimer may have one or more C-terminal modifications. The modifications may be the same or different. In one embodiment, the monomer subunits have C-terminal amide ends, where the charge is removed from the C-terminus of the peptide by amidation, especially when the peptide monomers are chemically synthesized. The uncharged C-terminal amide end more closely mimics the native protein, and therefore may increase the biological activity of the peptide.

In some embodiments, a monomer subunit of the peptide dimer may include additional residues at the C-terminal or the N-terminal. In some other embodiments, a monomer subunit of the peptide dimer may include a peptide tag at the C-terminal or N-terminal. In some embodiments, the tag may be an affinity tag such as a His-tag, Flag Tag, Twin-Strep Tag, etc.

Peptides described herein may also include, for example, biologically active mutants, variants, fragments, chimeras, and analogues. The term “fragments” encompasses amino acid sequences having truncations of one or more amino acids from the amino terminus (N-terminus), the carboxy terminus (C-terminus), or the interior of the peptide. Analogues of the invention are peptides with an insertion or a substitution of one or more amino acids. Variants, mutants, fragments, chimeras and analogues may function as inhibitors to abolish inhibitory effects of CSPGs on neural cells activated with CSPGs (without being restricted to the present examples).

In various nonlimiting embodiments, the peptide dimers disclosed herein may be used as therapeutic agents to promote cell growth, motility, survival and plasticity of these cells.

### Preparation of the Peptide Dimers

Embodiments of the present disclosure provide methods of preparing peptide monomers used for preparing the peptide dimers and methods of preparing the peptide dimers disclosed herein.

According to various embodiments, a peptide monomer used to prepare the peptide dimers may be prepared by methods known to those skilled in the art. For example, a peptide monomer can be prepared using conventional peptide synthesis techniques (e.g., solid phase synthesis) or molecular biology techniques.

In some embodiments, a transport moiety polypeptide as described above and a peptide domain described above may be separately synthesized and purified, and then non-covalently linked using a non-covalently linked polypeptide transduction agent such as that provided in the Chariot protein delivery system (See U.S. Pat. No. 6,841,535; J Biol Chem 274(35):24941-24946; and Nature Biotec. 19:1173-1176, all herein incorporated by reference in their entirety).

In some embodiments, a peptide monomer used for forming a peptide dimer disclosed herein may be produced by genetic engineering using a recombinant DNA. For example, a recombinant DNA may be engineered to encode a fusion peptide used for making a peptide dimer disclosed herein. The fusion peptide may comprise a peptide domain comprising an amino acid sequence selected from leukocyte antigen related (LAR) family phosphatase wedge domains or variants having at least 65% homologous thereto, and the peptide domain may be connected to a transport moiety disclosed herein via a cysteine residue or a peptide linker comprising one cysteine residue.

This recombinant DNA may be inserted in an expression cassette of an expression vector and operably linked to a regulatory region. The regulatory region typically comprises a promoter to regulate the expression of the peptide monomer in a cell carrying the vector. In some embodiments, the promoter is a constitutive promoter, such as a CMV, such that the peptide monomer may be expressed consistently in a cell carrying the vector. In some embodiment, the promoter is an inducible promoter, and the expression of the peptide monomer can be induced as needed.

In some embodiments, the vector is a plasmid vector and can be transformed into bacteria to store or to amplify, and can be transfected into mammalian cells to express the recombinant peptide.

In some embodiment, the preparation disclosed herein can include cultivating a host cell (bacterial or eukaryotic) under conditions, which provide for the expression of peptides and/or proteins within the cell.

The peptide monomer expressed in the host cell can be purified by affinity methods, ion exchange chromatography, size exclusion chromatography, hydrophobicity or other purification technique typically used for protein purification. The purification step can be performed under non-denaturing conditions. On the other hand, if a denaturating step is required, the protein may be renatured using techniques known in the art.

In some embodiments, the peptide monomers described herein can include additional residues that may be added at either terminus of a polypeptide for the purpose of providing a “linker” by which the polypeptides can be conveniently linked and/or affixed to other polypeptides, proteins, detectable moieties, labels, solid matrices, or carriers.

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In addition, a subject polypeptide can differ by the sequence being modified by terminal-NH2 acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

In some embodiments, the linker can be a flexible peptide linker that links the therapeutic peptide to other polypeptides, proteins, and/or molecules, such as detectable moieties, labels, solid matrices, or carriers. A flexible peptide linker can be about 20 or fewer amino acids in length. For example, a peptide linker can contain about 12 or fewer amino acid residues, e.g., 3, 4, 5, 6, 7, 8, 9, 10, 11, and 12. In some cases, a peptide linker comprises two or more of the following amino acids: glycine, serine, alanine, and threonine.

A peptide dimer disclosed herein may be prepared by dimerization of peptide monomers as prepared above, where the peptide monomers form the monomer subunits in the peptide dimer. The peptide monomers used to prepare a dimer may be identical or different, resulting in a homodimer or heterodimer, respectively.

In some embodiments, the peptide dimers are prepared by oxidation of a first peptide monomer and a second peptide monomer, such that the cysteines residues in the first peptide monomer and the second peptide monomer form a disulfide bond that connect the two peptide monomers together.

In one embodiment, the process of dimerization comprises combining a first peptide monomer and a second monomer in water, and either a) adding an oxidating agent and/or b) oxygenizing the solution to allow the formation of covalently cross-linked dimer. The first peptide monomer and the second monomer may be identical or different. In one embodiment, the combined concentration of the first and second peptide monomers in water may be at least 20 mg/mL or at least 40 mg/mL. In one embodiment, the combination of the first peptide monomer and the second monomer in water is held at a temperature of about 20° C. to about 25° C. In one embodiment, DMSO is added to the water before, during, or after the first peptide monomer and the second monomer are added to the water.

Oxidating agents used in the dimerization process may be chosen from any commonly oxidating agents. Nonlimiting example of oxidating agents may include bromates, chlorine oxyanion, chromates, hypoiodites, iodanes, iodates, interhalogen compounds, manganese compounds, nitrates, oxidizing acids, ozone, periodates, permanganates, peroxy acids, persulfates, ant rocket oxidizers. In some embodiments, an oxidating agent may be chosen form hydrogen peroxide, potassium dichromate, sodium or calcium hypochlorite, nitric acid, oxygen, ozone, potassium perchlorate, potassium chlorate, potassium permanganate, ammonium or sodium persulfate, or a combination thereof. In some embodiments, an oxidating agent is chosen from cupric sulfate, iodide, hydrogen peroxide, trans-3,4-dihydroxyselenolane oxide (DHS), supported methionine sulfoxide, N-Chlorosuccinimide (NCS), or a combination thereof.

In one embodiment, the oxidating agent is iodine, and the process employs microwave-assisted oxidation.

In some embodiments, a process for preparing the peptide dimers disclosed herein comprises mixing the first monomer and the second monomer in a solvent with cupric sulfate to produce the dimer. The solvent may further comprise purified water for purification (PWP). In one embodiment, the solvent may further comprise ethanol. In one embodiment, the pH of the mixture, i.e., dimerization reaction mixture, is maintained between about 8.5 and about 9.5.

An exemplary embodiment for preparing the peptide dimer disclosed here may include the following: 1) add PWP and ethanol into a oxidation beaker and stir for 15 minutes to make the mixture homogenous, the amounts of PWP and ethanol needed depending on the amount of peptide monomer; 2) after 15 minutes stirring, slowly add corresponding amount of the pure Compound 3 in the beaker and mix until all powder has been dissolved to form a clear solution, i.e., reaction mixture. Stir the reaction mixture for 15 minutes; 3) after 15 minutes stirring, check the initial pH of the reaction mixture and adjust the pH to about 9±0.5 using diluted ammonia solution, where the diluted ammonia solution is prepared by taking 100 mL of ammonia solution (25%) and diluted to 1L by PWP; 4) monitoring the pH of the reaction mixture hourly; if pH is lower than 9 0.5, adjust the reaction mixture by adding additional diluted ammonia solution; 5) After about 1 hour, add cupric sulfate to the oxidation reaction mixture periodically; 6) sample batch hourly and analyze reaction progress by HPLC, and allow the oxidation reaction to continue until free peptide monomer Compound 3 is detected by HPLC as less than 4±0.5%; 7) once the presence of Compound 3 is less than 4±0.5% as confirmed by HPLC, adjust the pH of the reaction mixture by adding acetic acid until the pH is about 4.5±0.5; 8) after the oxidation, the reaction mixture is adjusted to have a pH of about 4.5 0.5, and then filter the oxidation reaction mixture through 5-micron filter paper with the application of vacuum to the receiving container used for receiving the filtered solution; 9) collect the filtered solution and purify the filtered solution by preparative HPLC, 10) freeze dry the peak eluate from HPLC containing pure lyophilized peptide dimer compound.

### Formulations

Nonlimiting examples of materials that can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids; water; salts or electrolytes, such as protamine sulfate; disodium hydrogen phosphate; potassium hydrogen phosphate; sodium chloride; zinc salts; colloidal silica; magnesium trisilicate; polyvinyl pyrrolidone; polyacrylates; waxes; polyethylenepolyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols, such a propylene glycol or polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions.

Further, non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

According to various embodiments, the pharmaceutical compositions disclosed may be formulated in any suitable form for delivery to a subject in need thereof, either with fixed-dose or non-fixed dose. For example, the pharmaceutical composition may be adapted for oral or parenteral and may be administered to the subject in the dosage form of tablets, sugar-coated tablets, capsules, delayed-release hard capsules, softgel, chewable tablets, gummy, caplets, powders, granules, syrups, aerosols, inhalants, suppositories, solutions, suspensions, catheters containing the composition, syringes containing the composition, implants containing the composition, transdermal patch, or the like.

In some embodiments, the pharmaceutical compositions are formulated in liquid solution, typically in physiologically compatible buffers such as Hank's solution or Ringer's solution for injection. In some embodiments, a therapeutic agent comprising a peptide dimer or pharmaceutically acceptable analog, salt, or solvate thereof described herein may be formulated in solid form and re-dissolved or suspended immediately in a pharmaceutical acceptable solvent prior to use. For example, a peptide dimer or pharmaceutically acceptable analog, salt, or solvate thereof may be in lyophilized form that can be dissolved to obtain a final preparation to administer to a subject at the time of use. Injectable preparations (for example, sterile injectable aqueous or oleaginous suspensions) may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In some embodiments, the pharmaceutical composition does not comprise DMSO.

In order to prolong the effect of a drug, it is often desirable to slow the absorption of the drug from subcutaneous (s.c.) or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Absorption of a parenterally administered drug form can also be delayed by dissolving or suspending the drug in an oil vehicle.

In some other embodiments, the pharmaceutical compositions disclosed herein may be formulated in a dosage form such as a tablet, a softgel, a capsule, a caplet, a polypill, a chewable tablet, a gummy, a hard capsule, a transdermal patch, etc.

In some other embodiments, the pharmaceutical compositions disclosed herein may be formulated in liquid form for oral administration, where the pharmaceutical composition includes, among with the therapeutic agents (or active compounds), pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid compositions of a similar type may also be employed as fillers in soft and hard filled gelatin capsules using such excipients as lactose or milk sugar as well as high molecular weight polyethylene glycols and the like.

Active ingredients comprising the peptide dimers or pharmaceutically acceptable analog, salt, or solvate thereof can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets, and pills, the dosage forms may also comprise buffering agents.

The pharmaceutical compositions may be formulated in forms for topical or transdermal administration, where the forms may include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this disclosure.

The ointments, pastes, creams and gels may contain, in addition to an active compound of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this disclosure, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Methods of Treatment

The peptide dimers or pharmaceutically acceptable analog, salt, or solvate thereof, when being delivered to cell (such as neural cells), are able to promote cell growth, motility, survival and plasticity in the cells, e.g., neural cells.

As such, the peptide dimers or pharmaceutically acceptable analog, salt, or solvate thereof, and/or composition comprising the peptide dimers or pharmaceutically acceptable analog, salt, or solvate thereof can be used to abolish inhibitory effects of CSPGs on neural cells activated with CSPGs and promote cell growth, motility, and survival, and to treat diseases, disorders, and/or conditions associated with accumulation of CSPGs or with activation and signaling of LAR family of phosphatases.

Accordingly, embodiments of the present provide methods for treating diseases, disorders, and/or conditions associated with accumulation of CSPGs or with activation and signaling of LAR family of phosphatases using the peptide dimers or compositions comprising the peptide dimers discloses herein.

In various embodiments, provided include a method of treating a neurological condition, disease or disorder selected from the group consisting of neural injury, neurological disease caused by inflammation or autoimmunity, neurodegenerative disease, and a neurological condition, in a subject in need thereof, the method comprising administering an effective amount of a pharmaceutical composition disclosed herein to the subject.

In some embodiments, the neural injury is selected from the group consisting of acute neural injury, traumatic brain injury (TBI), spinal cord injury, concussion, stroke, including ischemic stroke, hemorrhagic stroke, and chronic stroke disease.

In some embodiments, the neurological condition, disease or disorder is selected from the group consisting of Alzheimer's Disease, dementias related to Alzheimer's Disease, Lewy diffuse body diseases, senile dementia, Parkinson's Disease, amyotrophic lateral sclerosis, multiple sclerosis (MS), optic neuritis, Huntington's Disease, Tourette's syndrome, hereditary motor and sensory neuropathy, diabetic neuropathy, progressive supranuclear palsy, Jakob-Creutzfeldt disease, epilepsy, and infectious disease.

Various embodiments further provide a use of a peptide dimer or a pharmaceutical acceptable analog, salt, or solvate thereof in the manufacture of a medicament for treatment of a neurological condition, disease or disorder selected from the group consisting of neural injury, neurological disease caused by inflammation or autoimmunity, neurodegenerative disease, a neurological condition, or a combination thereof.

### Administration and Dosages

In general, the pharmaceutical compositions disclosed herein may be administered to a subject in need thereof via any suitable route, including, for example, orally (e.g., in capsules, suspensions or tablets), systemically, or by parenteral administration. Non-limiting example routes include subcutaneous, intramuscular, intravenous, transdermal, intranasal, rectal, ocular, topical, sublingual, and buccal.

In one embodiment, the pharmaceutical compositions can be administered by lateral cerebroventricular injection into the brain of a subject, usually within 100 hours of when an injury (resulting in a condition characterized by aberrant axonal outgrowth of central nervous system neurons) occurs (such as within 6, 12, 24 or 100 hours, inclusive, from the time of the injury). The injection can be made, for example, through a burr hole made in the subject's skull. In another embodiment, the therapeutic agent can be administered through a surgically inserted shunt into the cerebral ventricle of a subject, usually within 100 hours of when an injury occurs (e.g., within 6, 12 or 24 hours, inclusive, from the time of the injury). For example, the injection can be made into the lateral ventricles, which are larger, even though injection into the third and fourth smaller ventricles can also be made. In yet another embodiment, the therapeutic agent can be administered by injection into the cisterna magna, or lumbar area of a subject, within 100 hours of when an injury occurs (such as within 6, 12, or 24 hours, inclusive, from the time of the injury).

In another embodiment, the pharmaceutical compositions can be administered to a subject at or near the site of injury, usually within 100 hours of when an injury occurs (e.g., within 6, 12, or 24 hours, inclusive, of the time of the injury). Such administration may optionally be subcutaneous.

In another embodiment, the pharmaceutical compositions can be administered to a subject at greater than 100 hours after the time of injury. In some cases, the administration will be a week after injury, multiple weeks after injury, or months or years after injury.

In certain embodiments, a therapeutic amount or dose of the therapeutic agents or compositions disclosed herein may range from about 0.1 mg/Kg to about 500 mg/Kg per body weight. In certain embodiments, a therapeutic amount or dose of the therapeutic agent or compositions disclosed herein may range from about 1 to about 50 mg/Kg. In general, treatment regimens according to the present disclosure comprise administration to a subject in need of such treatment from about 10 mg to about 1000 mg of the peptide dimers or pharmaceutically acceptable analog, salt, or solvate thereof of this disclosure per day in single or multiple doses. Therapeutic amounts or doses will also vary depending on route of administration, as well as the possibility of co-usage with other agents.

It will be understood, however, that the total daily usage of the compounds and compositions of the present disclosure will be decided by the attending physician within the scope of sound medical judgment. The specific inhibitory dose for any particular subject will depend upon a variety of factors including the disorder being treated and the severity of the disorder; the activity of the specific compound employed; the specific therapeutic agents or compositions employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts.

### Kits

One aspect of the present disclosure relates to a kit comprising a therapeutic agent comprising a peptide dimer or pharmaceutically acceptable salt or solvate thereof, or any composition comprising the peptide dimer or pharmaceutically acceptable analog, salt, or solvate thereof described above.

In some embodiments, the kit comprises one or more separate dosage forms, each dosage form comprises a pharmaceutical composition comprising an effective amount or one dose of a peptide dimer or pharmaceutically acceptable salt, analog, or solvate thereof disclosed herein for treating diseases, disorders, and/or conditions associated with inhibition of nervous system repair by chondroitin sulfate proteoglycans (CSPG) or with suppressive effects of LAR family phosphatases, such as PTPRD, PTPRF, and PTPRS on neuro repair.

In some embodiments, the kit may comprise a first container comprising a peptide dimer or pharmaceutically acceptable analog, salt or solvate thereof according to the present disclosure that is in free form, such as lyophilized powder, and optionally, a second container comprising a pharmaceutically acceptable solvent to dissolve the analog, salt, or solvate thereof. At the time of use, the peptide dimer or pharmaceutically acceptable analog, salt or solvate thereof according to the present disclosure in free form can be mixed with the solvent to produce a final preparation for administering to a subject in need thereof.

In some embodiments, the kit may further include an instruction for using the therapeutic agent or composition comprised in the kit for treating diseases, disorders, and/or conditions associated with inhibition of nervous system repair by chondroitin sulfate proteoglycans (CSPG) or with suppressive effects of LAR family phosphatases, such as PTPRD, PTPRF, and PTPRS on neuro repair.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The embodiments described herein are not limited to the particular methodology, protocols, and reagents, etc., and as such may vary. The terminology used herein is for the purpose of describing particular embodiments only and is not intended to be limiting in scope. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The following examples further illustrate aspects of the present disclosure. However, they are in no way a limitation of the teachings of the present disclosure as set forth. It should be understood that these Examples are given by way of illustration only. From the above discussion and these Examples, one of ordinary skill in the art can ascertain the essential characteristics of embodiments of the present invention. Without departing from the spirit and scope thereof, one skilled in the art can make various changes and modifications of the invention to adapt it to various usages and conditions. All publications, including patents and non-patent literature, referred to in this specification are expressly incorporated by reference herein.

### EXAMPLES

The practice of the present disclosure will employ, unless otherwise indicated, conventional techniques of organic synthesis, cell biology, cell culture, and molecular biology, which are within the skill of the art.

### Example 1: Synthesis and Purification of Peptide Dimer Compound 7

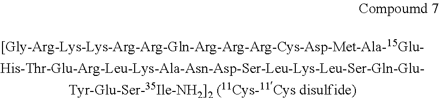

In this example, peptide dimer Compound 7, which comprises human PTPRS wedge domain, was prepared by oxidative dimerization of Compound 3 (peptide monomer of SEQ ID NO:3 (TAT-Cys-PTPRS human variant) following the raw materials and procedure described below. The average molecular weight of Compound 7 has 8575.65 Da.

### Raw Materials

Compound 3 (peptide monomer comprising amino acid sequence of SEQ ID NO:3, “TAT-Cys-PTPRS”): 11.5 gm
Purified water for purification (PWP): 1800 ml
Ethanol (AR): 200 ml
Ammonia solution: 21 ml, pH 9.9±0.5
Acetic acid (HPLC grade): 13 ml, pH 4.5±0.5

### Procedure of Preparation and Purification of a Peptide Dimer (Cupric Sulfate Protocol)

1) Add PWP and ethanol into an oxidation beaker and stir for 15 minutes to make the mixture homogenous, the amounts of PWP and ethanol needed depending on the amount of peptide monomer.
2) After 15 minutes stirring, slowly add corresponding amount of the pure Compound 3 in the beaker and mix until all powder has been dissolved to form clear solution. Stir the reaction mixture for 15 minutes.
3) After 15 minutes stirring, check the initial pH of the reaction and adjust the pH to about 9±0.5 using diluted ammonia solution, where the diluted ammonia solution is prepared by taking 100 mL of ammonia solution (25%) and diluted to 1L by PWP.
4) Monitoring the pH of the reaction mixture hourly. If pH is lower than 9 0.5, the adjust the reaction mixture by adding additional diluted ammonia solution.
5) After about 1 hour, add cupric sulfate to the oxidation reaction mixture periodically.
6) Sample batch hourly and analyze reaction progress by HPLC and allow the oxidation reaction to continue until free peptide monomer Compound 3 is detected by HPLC as less than 4±0.5%.
7) Once the presence of Compound 3 is less than 4±0.5% as confirmed by HPLC, adjust the pH of the reaction mixture by adding acetic acid until the pH is about 4.5±0.5.
8) After the oxidation reaction mixture is adjusted to have a pH of about 4.5±0.5, filter the oxidation reaction mixture through 5-micron filter paper with the application of vacuum to the receiving container used for receiving the filtered solution.
9) collect the filtered solution and purify the filtered solution by preparative HPLC.
10) freeze dry the peak eluate from HPLC containing pure Compound 7 to obtain lyophilized Compound 7.

Such obtained compound is an acetate salt of homodimer Compound 7, having identical monomer subunits comprising the amino acid sequence of SEQ ID NO:3.

Obtained peptide dimer is highly purified, with a purity of greater than 96.3%.

### Example 2: Preparation of Peptide Dimer Compound 8

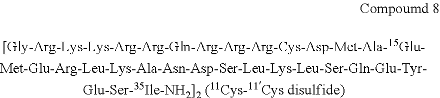

In this example, peptide dimer Compound 8, which comprises rats and mice PTPRS wedge domain, was prepared by oxidative dimerization of Compound 4 (peptide monomer of SEQ ID NO:4) (TAT-Cys-PTPRS rat variant) following the procedure described above in Example 1, except that the oxidation reaction continued until the free peptide monomer Compound 4 is detected by HPLC as less than 8±0.5%. The average molecular weight of Compound 8 is about 8635.84 Da.

### Raw Materials

Compound 4 (peptide monomer comprising amino acid sequence of SEQ ID NO:4, “TAT-Cys-PTPRS (rat)”): 1.1 gm
Ethanol (AR): 200 ml
Diluted ammonia solution: 440 ml, pH 9.9±0.5
Acetic acid (HPLC grade): 13 ml, pH 4.5±0.5

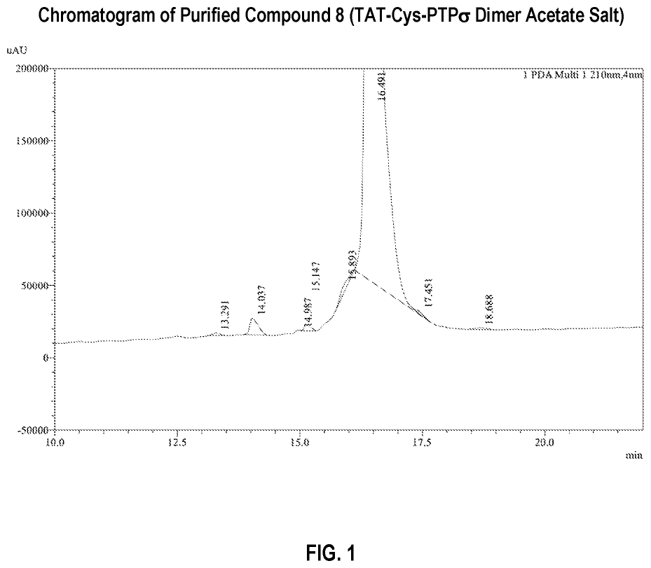

Such obtained compound is a homodimer of acetate salt of Compound 8, having identical monomer subunits comprising the amino acid sequence of SEQ ID NO:4. The purity of the purified Compound 8 salt was examined by chromatography. FIG. 1 is a plot illustrating the chromatogram showing the purity of obtained product. As can be seen in FIG. 1, obtained peptide dimer is highly purified, with a single peak corresponding to the purified peptide dimer, corresponding to a purity of greater than 96.3%.

### Example 3: Evaluation of the Efficacy of Compound 8 for Treating Spinal Cord Injury

### Objective

The goal of this study is to test the efficacy of a novel peptide dimer (Intracellular Sigma blocking peptide (ISP) dimer according to the present disclosure on behavioral changes in a rat model of thoracic traumatic Spinal Cord Injury (SCI).

### Materials And Methods

### Animals

Adult, female Lewis rats (~160 g on arrival) from Envigo were used as the subjects receiving the treatment in the study. The rats were assigned unique identification numbers (PGI ID and tail marks) and housed in ventilated cages. All rats were examined, handled, and weighed prior to initiation of the study to assure adequate health and suitability. During the course of the study, 12/12 light/dark cycles were maintained. The room temperature was maintained between 2° and 23° C. with a relative humidity maintained around 50%. Chow and water were provided adlibitum for the duration of the study. The rats were randomly assigned across treatment groups. Body weights were taken twice a week during the study.

### Formulations and Dosage

Lyophilized Compound 8 was dissolved in saline and injected s.c. at a volume of 500 μl to animal once daily from day 1-day 49 post spinal cord injury.

The dose concentration was 0.8 mg/ml, corresponding to 400 μg/rat. After reconstitutions, the solution was aliquoted and kept at −20° C. until dosing.

### Treatment Groups

12-13 rats were used in each of the following two groups:
1. Control group: SCI+Saline
2. Testing group: SCI+Compound 8 in Saline.

### Spinal Cord Injury and Post-Operative Care

Spinal cord injury (SCI) was introduced to the rats by surgery using the Infinite Horizon (IH) Impactor (200 kDyn force) described by Scheff et al. 2003. The surgery was performed with aseptic procedures in a designated area under deep anesthesia. Rats were anesthetized with isoflurane (4% for induction, 2% for maintenance), and O₂ (300 cm³/min) mixture. Hair was removed from the surgical site with clippers and the skin wase scrubbed with an antiseptic detergent. To maintain body temperature the animals were placed upon a homoeothermic blanket system. The T8 Vertebra of the rat was located externally, and an incision was made exposing the vertebral column from T6-T11. A laminectomy was performed using a surgical microscope to expose the dorsal spinal cord at the thoracic (T8) vertebral level. The vertebral column was stabilized by clamping the nearest rostral and caudal vertebral processes and the injury was produced using the IH impactor device at a 200 kDyn force. After each lesion the IH graphs and impact data were reviewed and recorded, rats presenting an impact force more than 10% higher or lower than expected and/or any abnormality on the force/displacement impact graphs were excluded from the study. Additionally, animals scoring more than 1 points in the Basso, Beattie and Bresnahan (BBB) score scale at 1 days after SCI were excluded for the study as well.

Following injury, muscles were closed in layers by the use of 4-0 Ethicon vicryl sutures, and the skin closed with wound clips which were removed 7-10 days after SCI.

After surgery, animals were kept in a warmed cage with water and food easily accessible. The rats received postoperative care that includes administration of antibiotics (Amoxicillin provided with the diet for 7-10 days); analgesics (Buprenorphine 0.03 mg/kg, SC for 2 days) and fluids (6-8 cc of lactated Ringer's solution, SC, twice a day for 3 days). Bladders were expressed twice a day until spontaneous voiding.

### Bladder Expression and Body Weights

Starting the day after lesion animals were observed twice a day and bladders were manually expressed using gentle abdominal pressure. Any abnormal appearance of the urine (i.e. cloudy urine, bloody urine) was recorded, and the size of the bladder estimated by trained researchers in order to obtain a categorical score (bladder score) (empty bladders=0; extra-small bladders=1; small bladders=2, medium bladders=3, large bladders=4). Urinary complications (bloody urine, foul-smelling urine, urine-stained fur) were reported and treated with SC fluids twice a day, and antibiotic treatment if the issues persisted. Bladder expression continue until spontaneous voiding was observed (empty bladder for 3 consecutive days).

Body weights were taken twice a week for the first 7 weeks after injury and then weekly until endpoint. Health and survival of the animals was evaluated during bladder expressions, body weight and behavioral test, any health concerns were reported and discussed with a veterinarian if needed.

### Basso, Beattie and Bresnahan (BBB) Locomotor Rating Scale

Locomotion changes were assessed using an open-field locomotor test (the Ohio State BBB Locomotor Rating Scale developed by Basso, et al. 1995). During each test, rats were observed moving around an empty wading pool for 4 minutes. Movements of joints in the hind limbs, weight support, and coordination between limbs were scored according to the BBB scale. This scale tracks the progressive recovery of hind-limb function following thoracic SCI. The scale can be divided into three parts reflecting the stages of recovery. Scores from 0 to 7 score the early phase of recovery with return of isolated movements of three joints (hip, knee, ankle); Scores from 8 to 13 describe the intermediate recovery phase with return of paw placement, stepping, and forelimb-hind limb coordination; and Scores from 14 to 21 rate the late phase of recovery with return of toe clearance during the step phase, predominant paw position, trunk stability, and tail position. BBB was assessed on days 1, 4 and 7 post injury and then weekly until completion of the study.

The BBB score scale obtained at day 1 after SCI was used to keep injury consistency. Prior to injury, all animals exhibited normal locomotion (21 score). At Day 1 after SCI, only animals scoring either 0 (no observable hind-limb movement) or 1 (slight movement of one or two hind-limbs joins) in the BBB scale were enrolled in the study and allocated into treatment groups. The treatments groups were balanced using the BBB score obtained at day 1 after SCI.

### Statistical Analysis

Data was analyzed by analysis of variance (ANOVA) followed by post-hoc comparisons where appropriate. An effect was considered significant if p<0.05. Data was represented as the mean and standard error to the mean (SEM).

### Results

### Body Weight

The effects of Compound 8 on body weights of the rats in the control group and in the testing group after SCI were measured twice a week for the first 7 weeks after injury and then weekly until the 13^{th} week. The results showed that there was no significant difference of body weights between the rats in the two groups and during the treatment period. The results indicate that the tested compound had no adverse effects on body weights during the course of the study.

### Basso, Beattie and Bresnahan (BBB) Locomotor Rating Scale

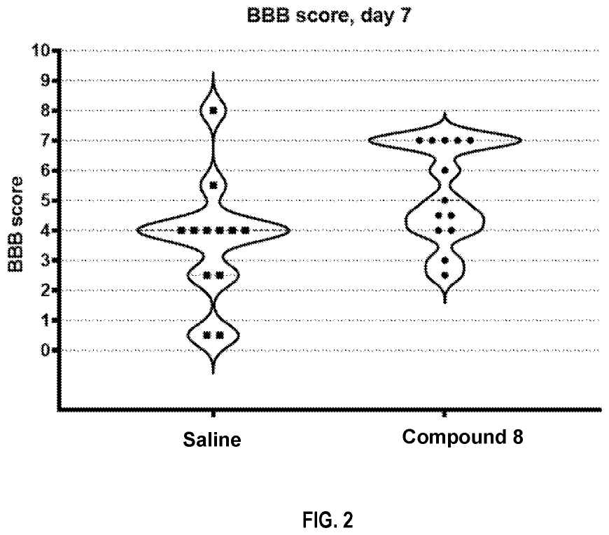

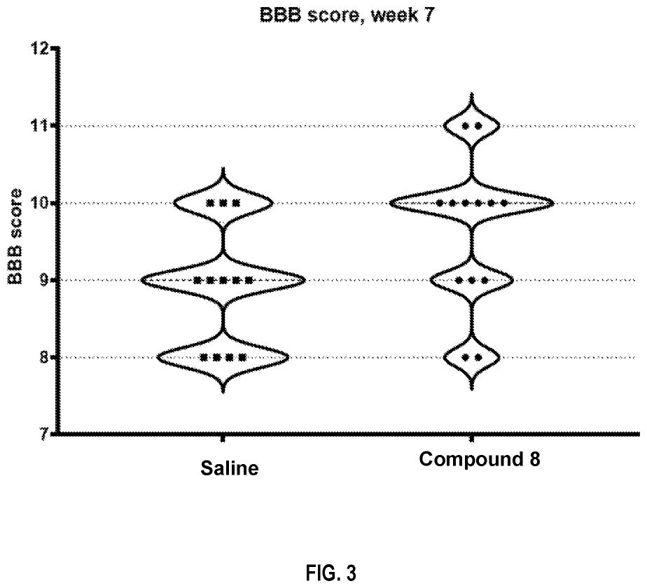

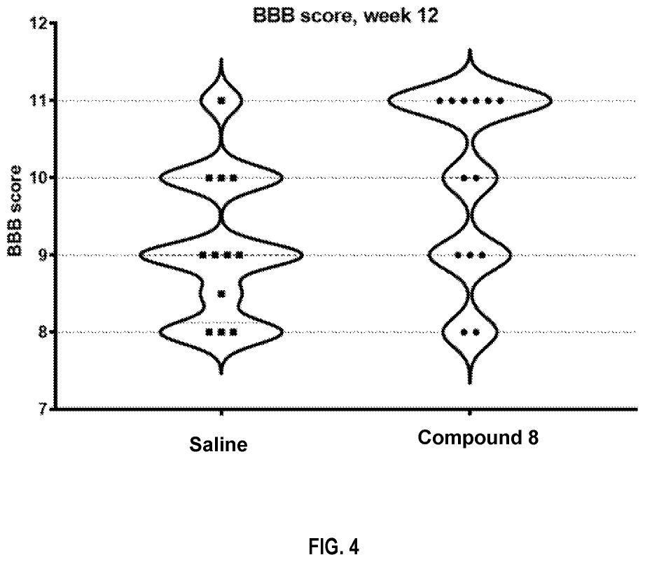

The effects of Compound 8 on open field locomotor performance evaluated using the BBB scale are shown in FIGS. 2-4, where FIG. 2 depicts the BBB score of the rats in the testing group compared to the control group at day 7 after SCI, FIG. 3 depicts the BBB score of the rats in the testing group compared to the control group at week 7 after SCI, and FIG. 4 depicts the BBB score of the rats in the testing group compared to the control group at week 12 after SCI. As can be seen in FIG. 2, at day 7 post-SCI, all 13 rats in the testing group that were treated with Compound 8 had BBB scores ranging from 2.5 to 7, with 6 rats having BBB scores of 6 to 7. In contrast, at day 7 post-SCI, 5 out of 12 rats in the control group that were treated with saline alone had a BBB score of 4, and 2 out of these 12 rats had a BBB score of about 0.5.

As shown in FIG. 3, at week 7 post-SCI, in the testing group, 6 rats had a BBB score of 10 and 2 rats had a BBB score of 11, while in the control group, majority rats had BBB scores of 8 to 9, and only 3 rats reach a BBB score of 10, where no rats in the control group reached a BBB score of 11.

As shown in FIG. 4, at week 12 post-SCI, most of the rats (8 out of 13) in the testing group that were treated with Compound 8 had BBB scores of about 10 or 11, while most of the rats (8 out of 12) in the control group that were treated with saline alone had BBB scores about 8 to 9.

The results demonstrated that Compound 8 promotes the recovery of movements of joints in the hind limbs, weight support, and coordination between limbs of SCI rats.

### Bladder Function Data

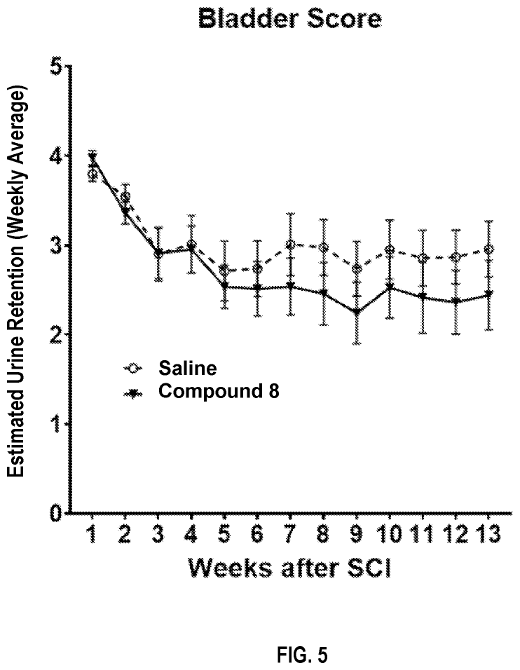

Following injury, animals exhibited loss of bladder function requiring manual bladder expression. During bladder expressions the size of each bladder was estimated by trained researchers and a semi-quantitative score was obtained. Daily estimates of urine retention were obtained by adding the bladder size scores of AM and PM bladder expressions. FIG. 5 illustrates the weekly average of urine retention in the rats in the testing group that were treated with Compound 8 in saline, compared to the rats in control group that were treated with vehicle control (saline).

It was observed that the rats treated with Compound 8 in saline showed a tendency to display smaller bladders during daily examinations and therefore less urine retention compared to the rats treated with vehicle control. As shown in FIG. 5, this trend was noticeable starting at week 4 after SCI. Most animals required bladder expressions once or twice a day during the complete length of the study, only 4 rats (1 from each experimental group) were able to recover spontaneous urine voiding by days 29 to 34 after SCI.

Urinary complications including bloody urine, foul-smelling urine, and urine-stained fur were monitored. When a new complication was observed after resolutions of a previous event, it was considered repetitive. Animals would be euthanized if the complications persisted after an additional cycle of antibiotics, when bladders could not be expressed or when general health deterioration was observed.

Urinary complications, such as repetitive bloody urine, urine-stained fur and urinary infections were observed, recorded, and treated, as needed. Table 7 summarizes the percentage of complications observed in each experimental group.

TABLE 7
Urinary complications identified during the course of the study

| | | Repetitive | Euthanasia due |
|---|---|---|---|
| Treatment | Urinary | Urinary | to Urinary |
| Groups | Complications | Complications | Complications |
| SCI + | 5 out of 12 | 3 out of 12 | none |
| Saline | 41% | 25% | |
| SCI + | 4 out of 13 | 1 out of 13 | none |
| Compound 8/ | 31% | 8% | |
| Saline | | | |

As can be seen in Table 7, urinary complications in rats receiving Compound 8/saline were less frequent than those in rats receiving the vehicle control.

### Summary

The acute administration Compound 8 had no adverse effect on the body weights of rats during the study. Bladder complications were less frequent in animals receiving Compound 8 when compared with animals receiving vehicle control. Animals receiving Compound 8 showed a decrease in the estimated size of bladders which was evident from week 8 to 13.

### Example 4—Effect of Compound 8 in Recovery of Walking in SCI Animals

In this experimental example, the effect of Compound 8 in recovery of walking in animal model of spinal cord injury was evaluated.

### Materials And Methods

### Animals

Adult, female Lewis rats (~160 g on arrival) from Envigo were used as the subjects receiving the treatment in the study. The rats were assigned unique identification numbers (PGI ID and tail marks) and housed in ventilated cages. All rats were examined, handled, and weighed prior to initiation of the study to assure adequate health and suitability. During the course of the study, 12/12 light/dark cycles were maintained. The room temperature was maintained between 2° and 23° C. with a relative humidity maintained around 50%. Chow and water were provided adlibitum for the duration of the study. The rats were randomly assigned across treatment groups. Body weights were taken twice a week during the study.

Spinal cord injury (SCI) was introduced to the animals and post-operative care was followed after the injury according to the methods described in Example 3.

### Formulations and Dosage

Lyophilized Compound 8 was dissolved in saline and injected s.c. at a volume of 500 μl to animal once daily from day 1-day 49 post spinal cord injury.

The target dose concentration was 0.8 mg/ml. After reconstitutions, the solution was aliquoted and kept at −20° C. until dosing.

### Treatment Groups

12-13 rats were used in each of the following two groups:
1) Control group: SCI+Saline
2) Testing group: SCI+Compound 8 in Saline.

### Evaluation of Occasional Walking and Frequent Walking

Inverse Kaplan-Meier graphs were generated based on the Basso, Beattie & Bresnaham (BBB) open field locomotor assessment described above in Example 3. A Kaplan-Meier graph is a statistical method to estimate the gain of a behavior (threshold) as a percent of the population over a given period of time. An animal reaching a threshold, as defined below, is indicated as an upward step with the Y axis denoting the accumulated percent of a group reaching the threshold, and the X axis indicating the time since SCI.

The BBB is an ordinal scale ranging from 0 to 21, with 0 indicating complete motor loss of the hind limbs and 21 indicating normal motor function. The BBB assesses the recovery to perform various locomotor movements, including the ability to bear weight, stand, walk, proper foot placement, and coordination. A step is defined as when a hind limb paw is in plantar contact with weight support then the hind limb is advanced forward and reestablishes plantar contact with weight support.

Recover occasional walking: Occasional walking is defined as less than or equal to half of the attempted steps are successful, corresponding to a BBB score of 10. The week post-SCI in which an animal achieved a 10 or above was recorded and plotted on the Kaplan Meier graph, as defined above.

Recover frequent walking: Frequent walking is defined as greater than half of the attempted steps are successful and corresponds to a BBB score of 11. The week post-SCI in which an animal achieved an 11 or above was recorded and plotted on the Kaplan Meier graph, as defined above.

Statistical methods: Kaplan Meier curves were analyzed using the log-rank test. Effects of Compound 4 and Compound 8 on the locomotor function and bladder function were assessed for statistical significance by repeated measures ANOVA (RM-ANOVA) with Geisser-Greenhouse correction for non-sphericity of data and Tukey correction for multiple comparisons or by nonparametric Friedman's test with Dunn's multiple comparison correction. The choice of RM ANOVA or Friedman's test for the analysis of each type of data (BBB scores, BBB subscores, and bladder scores) was determined by the normality of data distribution tests performed for each of the functional scores across all treatment cohorts. RM ANOVA was selected when the normality of data distribution test was passed by all three treatment cohorts of SCI rats (saline alone, Compound 4, and Compound 8). Friedman's test was used when at least one of the treatment cohorts failed the normality of data distribution test. Additional quantitative evaluation of the Compound 4 and Compound 8 treatment effects was performed by calculating and comparing percentages of animals achieving pre-specified thresholds of functional improvement in BBB scores, BBB subscores and bladder score.

### Results

### Compound 8 Improves Recovery of Occasional Walking in SCI Animals

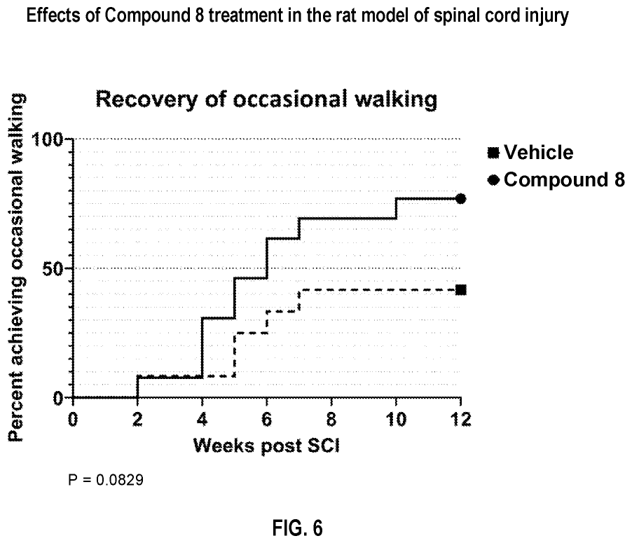

FIG. 6 is a graph depicting the recovery of occasional walking in Compound 8 treated SCI rats, compared to vehicle control treated SCI rats. As can be seen from FIG. 6, the SCI rats in both the testing group and the control group were not able to walk even occasionally during the first two weeks post the spinal cord injury. The SCI rats in both groups started to show certain degree of recovery of occasional walking (less than 10%). Starting from the fourth week post injury, consistent recovery of occasional walking was detected in the Compound 8 treated SCI rats, where the recovery reached 50% in less than 6 week and nearly 80% at week 12. In contrast, the recovery of occasional walking in vehicle control treated SCI rats was significantly slower compared to the recovery observed in Compound 8 treated SCI rats and had not reached 50% even at week 12.

The result demonstrates that Compound 8 provides effective treatment to spinal cord injury, significantly improving the recovery of occasional walking in animals of spinal cord injury.

### Compound 8 Improves Recovery of Frequent Walking in SCI Animals

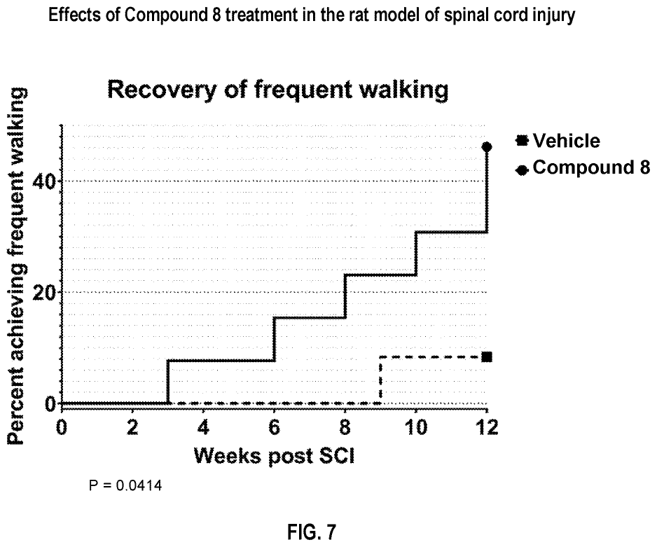

FIG. 7 is a graph depicting the recovery of frequent walking in Compound 8 treated SCI rats, compared to vehicle control treated SCI rats. As can be seen from FIG. 7, frequent walking in SCI rats was not detected during the first three weeks post the spinal cord injury. Recovery of frequent walking in Compound 8 treated SCI rats was detected after three weeks post injury, where the recovery was consistent and reached near 50% at week 12. In contrast, frequent walking was not detected in vehicle control treated SCI rats until week 9 post injury, with minimal improvement (10%) even after 12 weeks.

The result demonstrates that Compound 8 effectively improves the recovery of frequent walking in animals of spinal cord injury.

### Example 5—Comparison of BBB Sub-Scores of Animals Treated with Compound 8 and Compound 4

In this example, the effect of Compound 8 in improving the recovery of toe clearance, paw position, trunk stability and tail use independent of forelimb-hindlimb coordination was evaluated in comparison with Compound 4 (monomer of TAT-Cys-PTPRS rat variant) and saline.

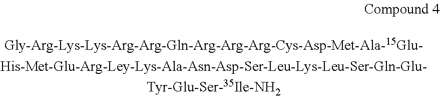

### Materials And Methods

Compound 4 was synthesized and HPLC purified with a purity of greater than 95%.

Animals were prepared according to the methods described above in Example 3.

Lyophilized Compound 8 and Compound 4 were separately dissolved in saline and injected s.c. at a volume of 500 μl to animal once daily from day 1-day 49 post spinal cord injury.

The target dose concentration was 0.8 mg/ml. After reconstitutions, the solutions were aliquoted and kept at −20° C. until dosing.

12-13 rats were used in each of the following three groups:
1) Control group: SCI+saline alone
2) Testing group: SCI+Compound 4 in saline
3) Testing group: SCI+Compound 8 in saline.

Spinal cord injury (SCI) was introduced to the animals and post-operative care was provided to the animals according to the methods described in Example 3.

### BBB Sub-Score Measurement

The BBB sub-scores quantify the recovery of toe clearance, paw position, trunk stability and tail use independent of forelimb-hindlimb coordination. BBB sub-scores were calculated using the observations recorded in the BBB records sheets. During BBB testing, the predominant position of the hind paws (parallel vs rotated), toe clearance, trunk stability and tail position (up, down, middle) were evaluated for animals reaching a BBB score of 10 or higher. Animals scoring less than 10 in BBB scale were considered to have BBB sub-score of “0”. Each rat could achieve a maximum sub-score of 13 using the following scale shown in Table 8.

TABLE 8
Locomotor features considered for the BBB sub-score calculation

| | | |
|---|---|---|
| BBB sub-score | Left | Right |

| | | | |
|---|---|---|---|
| Parallel paw position | At initial contact | 1 | 1 |
| (up to 4 points) | At lift off | 1 | 1 |
| Toe clearance | Occasional | 1 | 1 |
| (up to 6 points) | Frequent | 2 | 2 |
| | Consistent | 3 | 3 |

| Trunk Stability (1 point) | | | 1 |
|---|---|---|---|
| | Tail position | Middle | 1 |
| | (up to 2 points) | Up | 2 |

Total (Up to 13 points)

### Results

As described above in Example 3, the upper points on the BBB scale (14-21) quantify fine motor movements associated with hind limb locomotor including toe clearance, predominant paw position, trunk stability, and tail position. These fine motor movements are only considered if animals display consistent coordination. A BBB sub-score was applied to quantify these improvements independent of coordination.

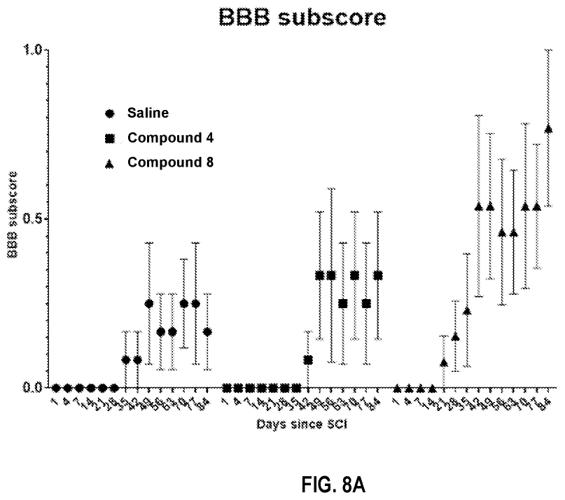

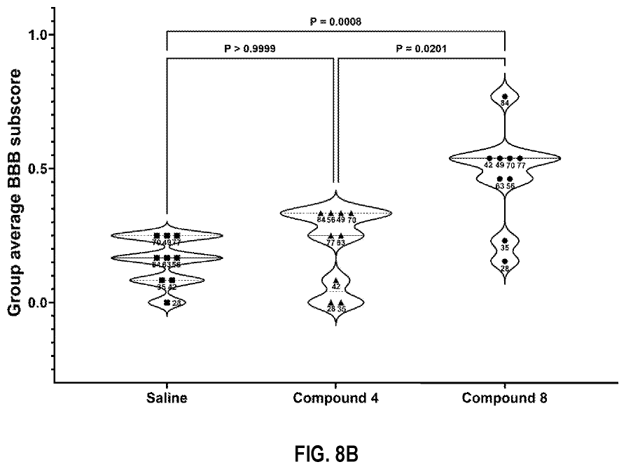

FIGS. 8A and 8B depict the effects of Compound 8, compared to Compound 4 and saline control, in improving the recovery of fine motor movements associated with hind limb toe clearance, paw position, trunk stability, and tail position in SCI animals as measured by BBB sub-score. FIG. 8A data are presented as mean±SEM. N=12-13/group. As can be seen in FIG. 8A, after SCI, all rats presented with a BBB sub-score of 0 during the first 14 days of the behavioral evaluation, indicating that there was no recovery during the first 14 days. Treatment with compound 8 resulted in accelerated onset of functional improvement when compared to the corresponding effects of the vehicle control (saline alone) and Compound 4. The onset of functional improvement was observed on day 21 in the Compound 8 cohort of SCI rats, on day 42 in the Compound 4 cohort, and on day 35 in the control (saline alone) cohort. A sustained trend of continued functional improvement was apparent in the cohort of rats treated with Compound 8 and was not apparent in the control (saline alone) or Compound 4 cohorts.

FIG. 8B depicts violin plots presenting distributions of the weekly average BBB sub-scores in the saline alone, Compound 4, and Compound 8 treatment cohorts of SCI rats. Each data point represents a weekly average BBB sub-score in the saline alone (control), Compound 4, and Compound 8 treatment cohorts as annotated on the X-axis. The numbers adjacent to the data points indicate the numbers of days post-SCI at which individual and treatment cohort weekly average BBB sub-scores were calculated. Statistical comparison of the treatment effects was performed using Friedman's test (repeated measures ANOVA for nonparametric values) with Dunn's correction for multiple comparisons and p-value of less than 0.05 defined as the criterion of statistical significance. As can be seen in FIG. 8B, treatment of SCI rats with Compound 8 produced a statistically significant effect on BBB sub-score when compared to the effect of control vehicle (saline alone) (p=0.0008) or to the effect of Compound 4 (p=0.0201). No statistically significant difference (p>0.9999) was observed between the cohort treated with Compound 4 and the cohort treated with saline alone (control).

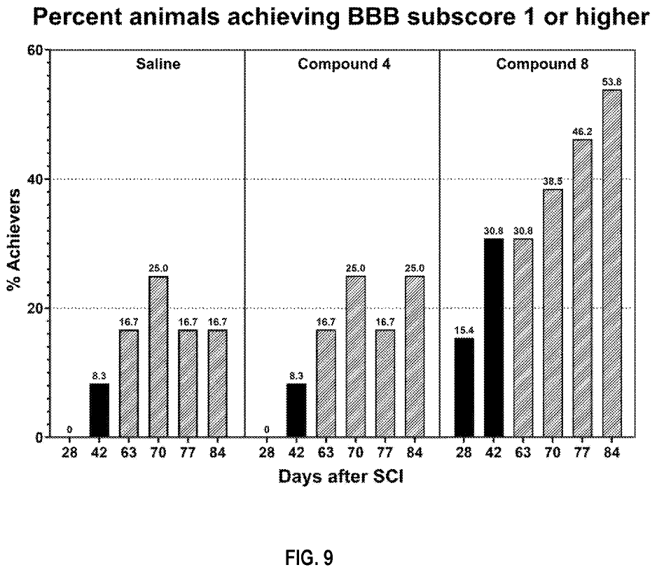

FIG. 9 further depicts the percentages of SCI rats achieving a BBB sub-score of 1 or higher when treated with Compound 8 in comparison with the SCI rats treated with Compound 4 or saline alone. The solid bars represent data corresponding to the period of saline, Compound 4, or Compound 8 treatment administration. The striped bars represent data collected after discontinuation of treatment. As can be seen in FIG. 9, at each time point post-SCI, e.g., day 28, 42, 63, 70, 77, and 84, the percentage of the rats achieving BBB sub-score of 1 or better (percentage of achievers) was higher in the group treated with Compound 8 compared to the groups treated with Compound 4 or saline alone. Treatment with Compound 8 resulted in continued functional improvement after discontinuation of Compound 8 administration. In contrast, no similar effect was observed in the cohorts of SCI rats treated with Compound 4 or with saline alone.

### Example 6—Pharmacokinetic Profiles of Compound 7

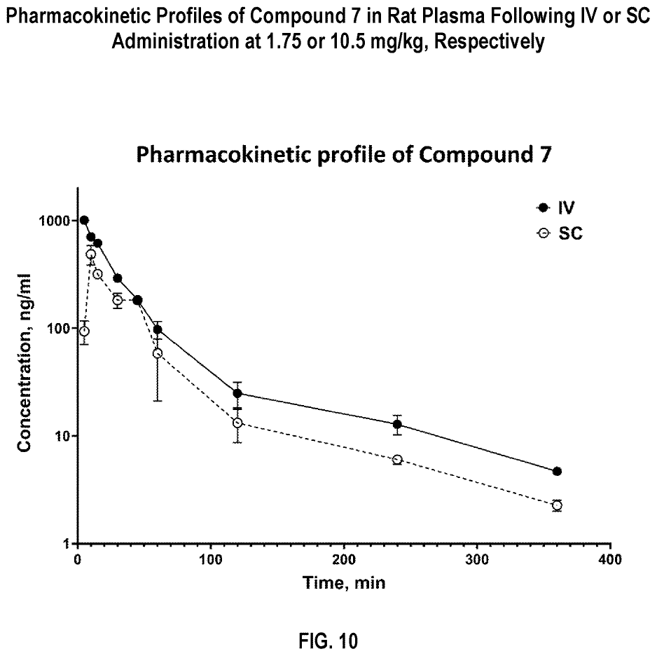

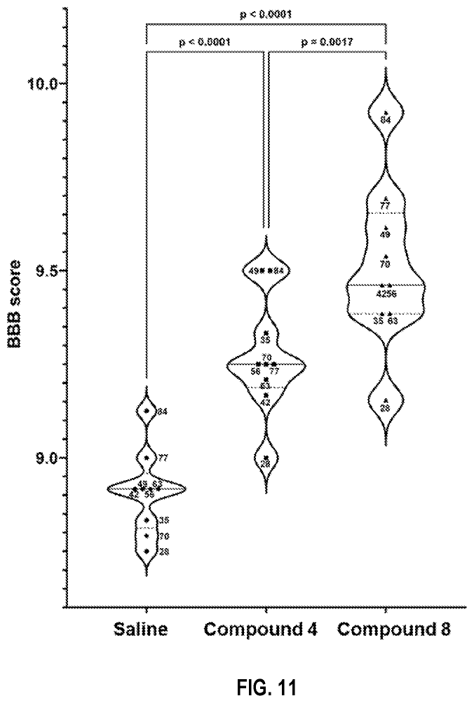

Pharmacokinetic profiles of the Tat-Cys-PTPRS wedge domain dimer in rat plasma following intravenous or subcutaneous administration were studied using Compound 7 as an example. In this study, Compound 7 in saline as prepared according to Example 3 was respectively administered to three rats intravenously at a dose of 1.75 mg/kg (body weight of the rat) and respectively administered to another three rats subcutaneously at a dose of 10.5 mg/kg (body weight of the rat). The concentrations of Compound 7 in the rat plasma following the administration were measured. The results are illustrated in FIG. 10, which shows the pharmacokinetic profiles of the Tat-Cys-PTPRS wedge domain dimer (Compound 7) in rat plasma following intravenous or subcutaneous administration at 1.75 or 10.5 mg/kg, respectively.

### Example 7—Comparison of BBB Scores and Bladder Scores of Animals Treated with Compound 8 and Compound 4

In this study, the effects of Compound 8 in improving the recovery of open field locomotor performance and bladder function in the SCI rats were further evaluated in comparison with Compound 4 by assessing the BBB scores and bladder scores according to the methods described in Example 3.

In this assessment, 12-13 rats were included in each of the following groups (1) Control group 1: SCI+saline alone; (2) Control group 2: SCI+Compound 4 in saline; (3) Testing group: SCI+Compound 8. The treatment was started on day 1 post-injury and continued through (last dose administered on) day 49 post-injury. The post-treatment observation period commenced on day 50 post-injury and continued until the end of study on day 84 post-injury.

The results of the BBB score assessment are shown in FIGS. 11, 12, and 13. FIG. 11 depicts violin plots presenting distributions of the weekly average BBB scores in each of the three treatment cohorts, as annotated on the X-axis. Each data point represents a weekly average BBB score in the saline alone (control), Compound 4, and Compound 8 treatment cohorts as annotated on the X-axis. The numbers adjacent to the data points indicate the numbers of days post-SCI at which individual and treatment cohort weekly average BBB scores were calculated. Statistical comparison of the treatment effects was performed using repeated measures ANOVA with Tukey correction for multiple comparisons and p-value of less than 0.05 defined as the criterion of statistical significance.

The results of the BBB score assessment are shown in FIGS. 11, 12, and 13. FIG. 11 depicts violin plots presenting distributions of the weekly average BBB scores in each of the three treatment cohorts as annotated on the X-axis. Each data point represents a weekly average BBB score in the saline alone (control), Compound 4, and Compound 8 treatment cohorts as annotated on the X-axis. The numbers adjacent to the data points indicate the numbers of days post-SCI at which individual and treatment cohort weekly average BBB scores were calculated. Statistical comparison of the treatment effects was performed using repeated measures ANOVA with Tukey correction for multiple comparisons and p-value of less than 0.05 defined as the criterion of statistical significance.

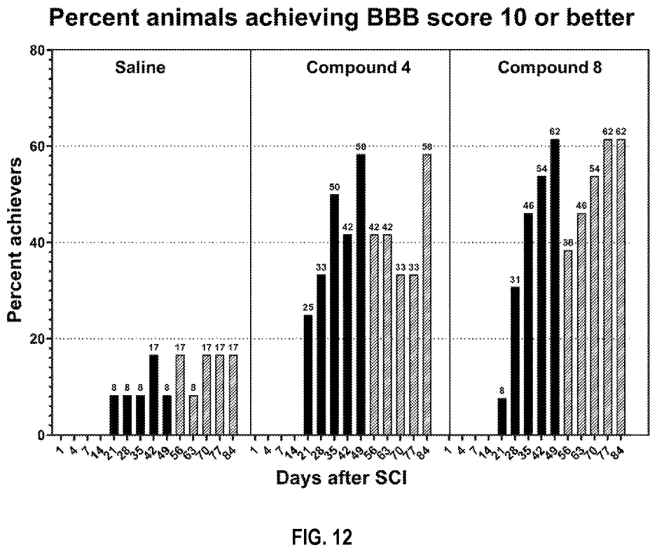

As can be seen in FIG. 11, the rats treated with Compounds 4 and 8, exhibited higher BBB scores, compared to the rats that were treated with saline alone. Compound 4 and Compound 8 each produced significant improvement of the locomotor performance measured with the BBB scores. The corresponding p-values were as follows: p<0.0001 for the comparison of Compound 4 vs. saline alone SCI rat cohorts; p<0.0001 for the comparison of Compound 8 vs. saline alone SCI rat cohorts. Additionally, Compound 8 was significantly (p=0.0017) more efficacious than Compound 4 in improving the BBB scores than Compound 4. FIG. 12 depicts the percentages of rats in each group that exhibited BBB scores of 10 or above post-SCI. The solid bars represent data corresponding to the period of test article (saline alone, Compound 4, or Compound 8) administration. The striped bars represent the data collected during the period of observation after discontinuation of the active treatment with saline alone, Compound 4, or Compound 8. The results show that during day 28 to day 84 of the treatment post-SCI, less than 20% of the SCI rats treated with saline alone had BBB scores of 10 or above, while about 30% to about 60% of the SCI rats treated with Compound 4 and Compound 8 achieved BBB scores of 10 or above. The overall percentages of rats achieving BBB scores of 10 or above during the treatment were higher in group (3), compared to the rats in group (2). More than half of the rats in group (3) achieved BBB scores of 10 or above after 70 days post the SCI.

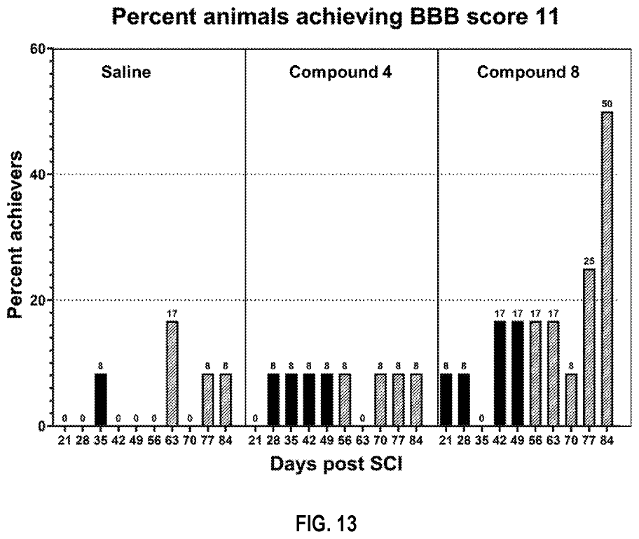

FIG. 13 depicts the percentages of rats in each group that exhibited BBB scores of 11 post-SCI. The solid bars represent data corresponding to the period of test article (saline alone, Compound 4, or Compound 8) administration. The striped bars represent the data collected during the period of observation after discontinuation of the active treatment with saline alone, Compound 4, or Compound 8. As can be seen in FIG. 13, more than 20% and about 50% of the rats treated with Compound 8=achieved a BBB score of 11 on day 77 and day 84, respectively. These results were not observed in rats treated with saline alone or with Compound 4 and demonstrate that Compound 8 was more effective than Compound 4 in promoting the recovery of locomotor function assessed using the BBB scoring metric.

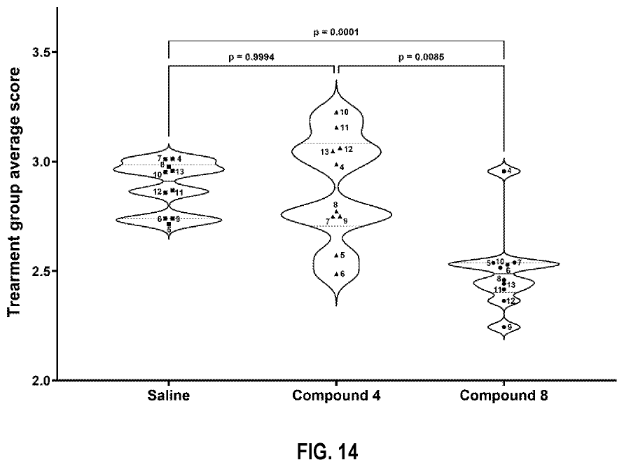

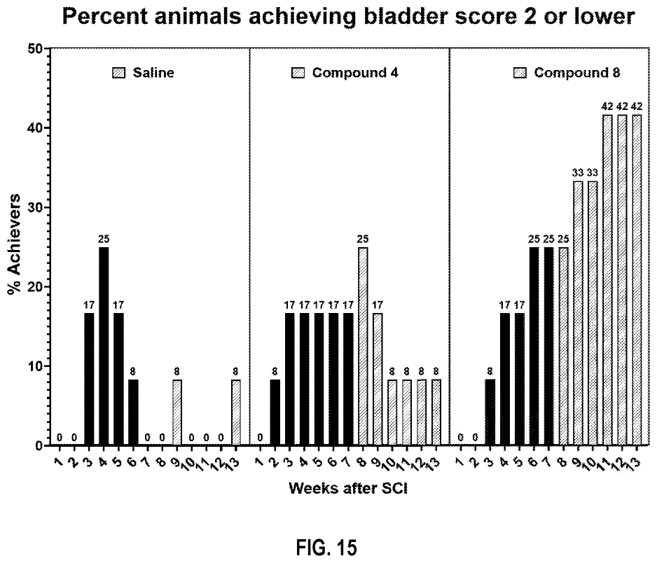

The results of the bladder score assessment are illustrated in FIGS. 14 and 15. FIG. 14 depicts violin plots presenting distributions of the weekly average bladder scores in each of the three treatment cohorts of SCI rats, as annotated on the X-axis. Each data point represents a weekly average bladder score in the saline alone (control), Compound 4, and Compound 8 treatment cohorts as annotated on the X-axis. The numbers adjacent to the data points indicate the numbers of days post-SCI at which individual and treatment cohort weekly average bladder scores were calculated. Statistical comparison of the Compound 4 and Compound 8 treatment effects was performed using repeated measures ANOVA with Tukey correction for multiple comparisons and p-value of less than 0.05 defined as the criterion of statistical significance. As can be seen in FIG. 14, the average weekly bladder scores of the rats treated with Compound 8 were significantly lower than the scores of the rats treated with either Compound 4 or saline alone. (in FIG. 14: p=0.0001 between saline and Compound 8 treated groups, and p=0.0085 between Compound 8 and Compound 4 treated groups). This result indicates that the rats treated with Compound 8 had a tendency to display smaller bladders during daily examinations and therefore less urine retention compared to the rats treated with Compound 4 or saline alone.

The percentages of rats achieving a bladder score of 2 or less (better) during the treatment post-SCI are illustrated in FIG. 15 The solid bars represent data corresponding to the period of test article (saline alone, Compound 4, or Compound 8) administration. The striped bars represent the data collected during the period of observation after discontinuation of the active treatment with saline alone, Compound 4, or Compound 8. The results show that overall more rats in the group treated with Compound 8 showed better bladder scores than the rats treated with saline alone or Compound 4. About 25% of rats treated with Compound 8 exhibited bladder scores of 2 or better starting on week 6 post-SCI. The bladder scores of the rats treated with Compound 8 kept improving from week 8 to week 13, with more than 40% of rats achieving a score of 2 or better during weeks 10 to 13. These results were not observed in the rats treated with saline alone or Compound 4.

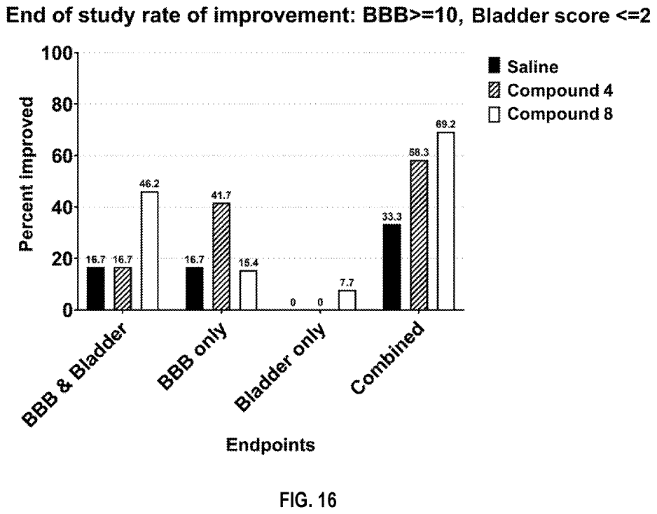

At the end of the study, the performance of Compound 8 was evaluated in comparison with Compound 4 and saline alone by calculating the percentages of animals achieving predefined thresholds of functional improvement in the following categories: (i) Simultaneous improvement in BBB and bladder scores, (ii) Improvement in the locomotor function (BBB score) alone but not the in the bladder score, (iii) Improvement in the bladder score alone but not in the BBB score. FIG. 16 depicts the percentage of rats in each of these three categories, as annotated on the X-axis. An additional group (annotated as “Combined”) represents the combined percentage of rats achieving improvement in any of the first three categories for each treatment cohort (saline alone, Compound 4, and Compound 8). The predefined thresholds of functional improvement were set at BBB score of at least 10 and bladder score of 2 or lower at the end of the study (study day 84).

As shown in FIG. 16, nearly 50% of SCI rats treated with Compound 8 achieved BBB scores of at least 10 and bladder scores of 2 or less simultaneously, while only about 17% of rats treated with saline alone or Compound 4 achieved this combined improvement at the end of the study. Improvement in the BBB score only (with no bladder score improvement) was observed in about 40% of the rats treated with Compound 4, compared to about 17% of rats treated with saline only and about 15% of the rats treated with Compound 8. In addition, about 8% of rats treated with Compound 8 showed improvement in bladder-only scores (reduced the bladder scores). In contrast, the rats treated with Compound 4 showed no improvement in bladder-only scores only, compared to the rats treated with Compound 8. The combined rates of the functional improvement (the simultaneous BBB score and bladder score improvement, BBB score only, and bladder score only percentages combined) were about 33%, about 60%, and about 70% in the SCI rats treated with saline alone, Compound 4, or Compound 8, respectively.

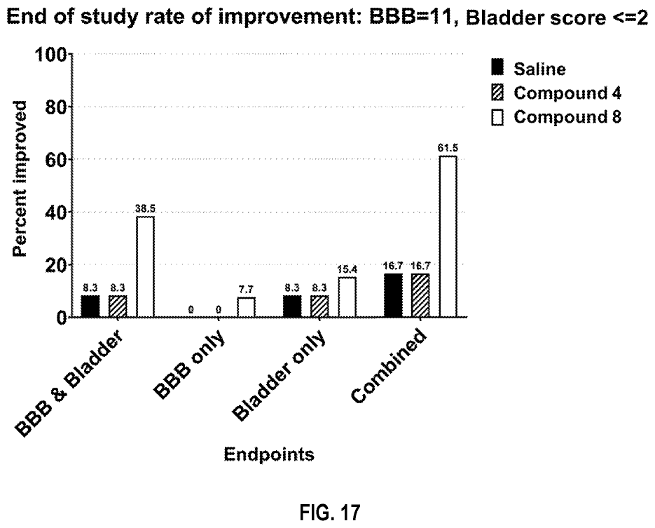

FIG. 17 depicts the percentage of rats that reached BBB scores of 11 and/or bladder scores of 2 or less at the end of the study. These results further demonstrate that Compound 8 but not Compound 4 predominantly caused simultaneous improvement in BBB and bladder scores, alone. At the end of the study, about 40% of SCI rats treated with Compound 8 achieved simultaneous improvement at the levels of BBB score of 11 and BL scores of 2 or less, whereas only about 8% of the rats achieved this simultaneous improvement in each of the saline alone and Compound 4 cohorts. No SCI rats reached BBB score of 11 alone in the saline only and Compound 4 cohorts of SCI rats vs. about 8% in the Compound 8 cohort. The combined rate of functional improvement (the combined percentages of SCI rats achieving BBB score of 11 only, bladder score of 2 or lower only, and BBB score of 11 and bladder score of 2 or lower simultaneously) was about 60%/ This rate of functional improvement was not observed in rats treated with saline alone or Compound 4.

Taken together, the results presented on FIGS. 16 and 17 show that Compound 8 was more effective than Compound 4 in promoting simultaneous improvement in both the locomotor (BBB score) and bladder (bladder score) functions, while Compound 4 effect was predominantly limited to the improvement in locomotor function only and lower in magnitude (maximum BBB score of 10 in the Compound 4 cohort vs. 11 in the Compound 8 cohort).

### Example 8—In Vitro Stability of Compound 3 and Compound 7 in Simulated Biological Matrices (Buffered Saline and Plasma)

Stability of Compound 7 (human variant) in physiological buffer (Hanks Balanced Salt Solution, HBSS) and in plasma was evaluated in comparison with Compound 3.

Compound stability was defined as % recovery of the compound after 5 min, 15 min, and 30 min of incubation in the presence of corresponding plasma.

### Materials And Methods

200 μL of 1.6 μM Compound 3 and Compound 7 solutions were incubated in the matrix (Hank's Balanced Salt Solution or plasma) at 37° C. for 0, 5, 15, or 30 minutes. The incubation was followed by addition of 200 μL acetonitrile, vortexing and subsequent acidification with 45 μL of pure acetic acid. After additional vortexing, the resulting mixture was centrifuged for 5 min at 14,000 rpm and the supernatant was collected for analysis.

Determination of the Compound 3 or Compound 7 levels in the supernatant samples prepared as described above was performed using an LC-MS/MS method as follows: 40 μL of 5 μM Compound 7 in 10% acetic acid aqueous solution was added to 160 μL of the supernatant. 20 μL of 5 μM Compound 3 in 10% acetic acid aqueous solution was added to 180 μL of the supernatant. The processed sample (20 μL) was injected onto an ACE Excel 2 C18 column (3.0 mm×75 mm, 2.0 μm).

Percentages of Compound 3 and Compound 7 recovery after 0 minutes (at the start of the experiment), 5 minutes, 15 minutes, and 30 minutes incubation time points were calculated by dividing the corresponding peak areas by the peak area obtained from the samples prepared with no incubation (0 minutes of incubation time). The recovery percentage at time 0 was defined as 100% recovery.

Specific presence of Compound 3 or Compound 7 in the chromatographic peak fractions was verified using Agilent 1200 high performance liquid chromatography (HPLC) system coupled with a Sciex API 3200 triple quadrupole mass spectrometer.

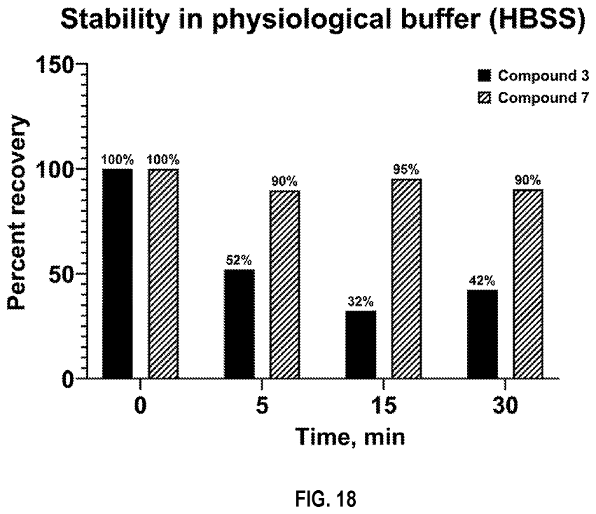

FIG. 18 depicts the stability of Compound 7 in physiological buffer (HBSS), compared with Compound 3. It can be seen that Compound 7 was highly stable in HBSS, with 90% recovery for at least 30 minutes of incubation with HBSS. In contrast, Compound 3 was less stable in HBSS, with about 42% recovery at 30 minutes.

### Results

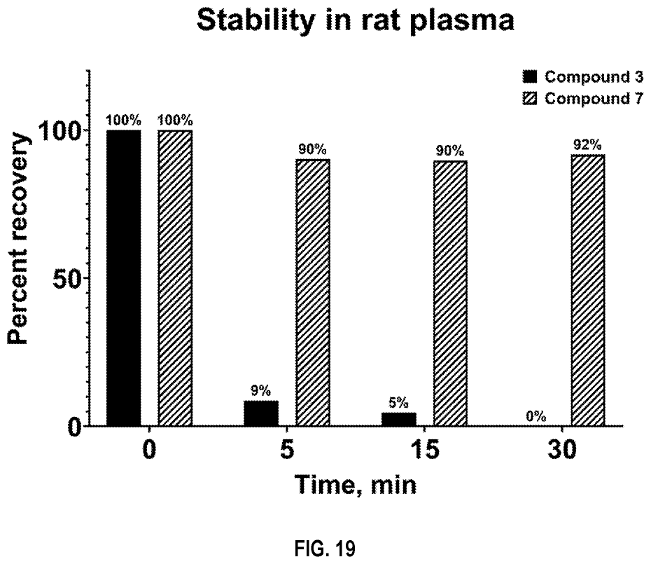

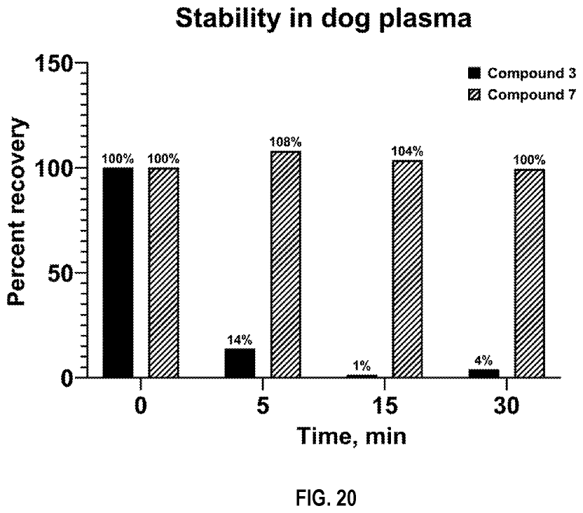

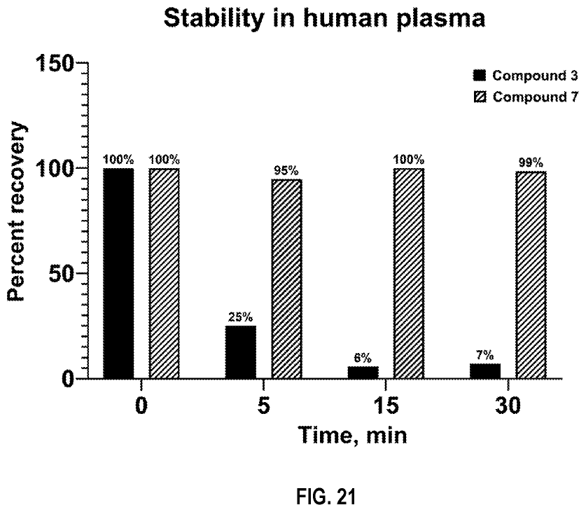

FIGS. 19, 20, and 21 depict the stabilities of Compound 7 in rat plasma, dog plasma, and human plasma, respectively, compared to Compound 3. The results show that Compound 7 was highly stable in the plasma, with a recovery rate of 92% in the rat plasma, 100% in the dog plasma, and 99% in the human plasma after 30 minutes of incubation. In contrast, Compound 3 exhibited low stability in the plasma.

### Example 9—Distinct Colloidal Behavior of Compound 7 and Compound 3

The patterns of self-assembly of Compound 7 and Compound 3 were analyzed by negative stain transmission electron microscopy.

### Materials And Methods

Compound 3 and Compound 7 were dissolved in sterile water or in 0.9% (w/v) sodium chloride in sterile water to obtain a 20 mg/mL solution. Formvar/carbon coated grids were exposed to 15 mL drops of the Compound 3 or Compound 7 solutions for approximately 1 minute, blotted and then exposed to 15 mL drops of sterile water for 10 seconds, blotted again and then stained with 2% (w/v) aqueous uranyl acetate for 30 seconds, The stained samples were exposed to 15 mL drops of sterile water for 10 seconds and blotted dry prior to examination by transmission electron microscopy.

### Results

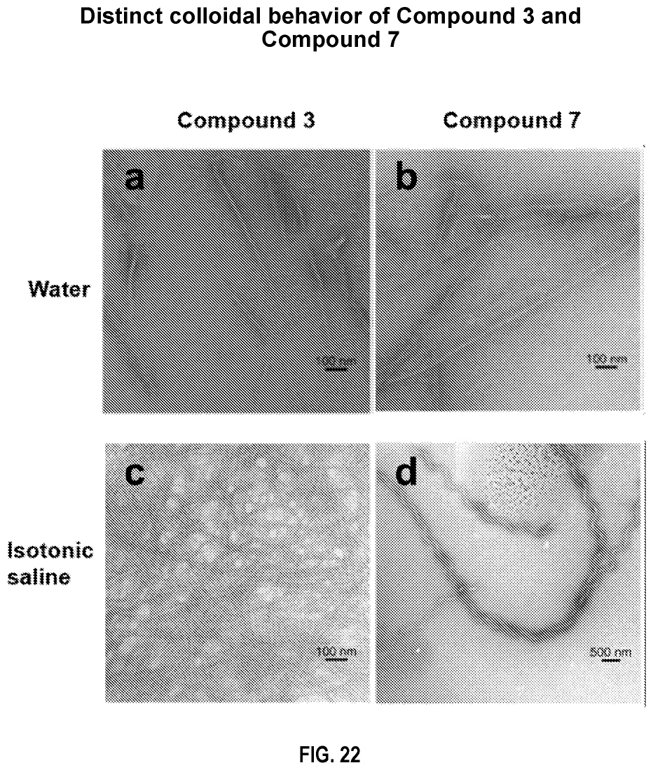

FIG. 22 presents a set of transmission electron microscopy (TEM) images showing colloidal structures resulting from self-assembly of Compound 3 (panels “a” and “c”) and Compound 7 (panels “b” and “d”) in water (panels “a” and “b”) and in isotonic saline (panels “c” and “d”). The images presented in panels “a” and “b” demonstrate formation of similar rod-shaped nanostructures by both Compound 3 and Compound 7. The images presented in panels “c” and “d” demonstrate the formation of an amyloid-like fibrillar meshwork by Compound 3 whereas Compound 7 formed distinct, larger-in-size helical colloidal particles not observed with Compound 3. These data illustrate the different physicochemical behavior of Compound 3 compared to Compound 7 in the simulated biological matrix (isotonic saline).

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A pharmaceutical composition comprising a peptide that is a dimer comprising a first monomer covalently cross-linked with a second monomer, each monomer comprising a domain comprising an amino acid sequence derived from a cytoplasmic wedge domain of a receptor-type protein-tyrosine phosphatase (PTPR), and wherein the mass ratio of the dimer to free monomer in the pharmaceutical composition is greater than 1:20.

2. The pharmaceutical composition of claim 1, wherein the ratio is greater than 1:1.

3. The pharmaceutical composition of claim 1 or claim 2, wherein the ratio is greater than 4:1.

4. The pharmaceutical composition of any one of claims 1-3, wherein the ratio is greater than 10:1.

5. The pharmaceutical composition of any one of claims 1-4, wherein the dimer comprises a transport moiety attached to the domain via one cysteine residue or a peptide linker comprising one cysteine residue.

6. The pharmaceutical composition of claim 5, wherein the transport moiety comprises a TAT sequence.

7. The pharmaceutical composition of claim 6, wherein the TAT sequence comprises an amino acid sequence that is at least 70% identical to the amino acid sequence of SEQ ID NO:5, 6, or 7.

8. The pharmaceutical composition of any one of claims 1-7, wherein the dimer improves neural cell repair.

9. The pharmaceutical composition of any one of claims 1-7, wherein the domain is selected from the group consisting of a PTPRF wedge domain, a PTPRD wedge domain, and a PTPRS wedge domain, and variants having at least 70% identity thereto.

10. The pharmaceutical composition of any one of claims 1-9, wherein the domain comprises an amino acid sequence that is at least 70% identical to the amino acid sequence of SEQ ID NO:8, 9, 10, or 11.

11. The pharmaceutical composition of any one of claims 1-10, wherein the first monomer and the second monomer each independently comprise:
a first domain comprising an amino acid sequence that is at least 70% identical to the amino acid sequence of SEQ ID NO:5, 6, or 7;
a second domain comprising an amino acid sequence that is at least 70% identical to the amino acid sequence of SEQ ID NO:8, 9, 10, or 11; and
a cysteine residue;
wherein the dimer comprises a chemical linker or bond between the cysteine residue of the first monomer and the cysteine residue of the second monomer.

12. The pharmaceutical composition of claim 11, wherein the first domain comprises an amino acid sequence that is identical to the amino acid sequence of SEQ ID NO:5, 6, or 7.

13. The pharmaceutical composition of claim 11 or claim 12, wherein the first domain comprises an amino acid sequence that is identical to the amino acid sequence of SEQ ID NO:5.

14. The pharmaceutical composition of any one of claims 11-13, wherein the second domain comprises an amino acid sequence that is identical to the amino acid sequence of SEQ ID NO:8, 9, 10, or 11.

15. The pharmaceutical composition of any one of claims 11-14, wherein the dimer has the following structure: 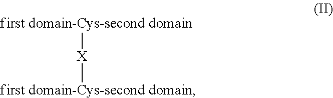
wherein X is a bond or a chemical linker between the two cysteine residues.

16. The pharmaceutical composition of claim 15, wherein X is a chemical linker chosen from a disulfide bond, a thioether bond, or a thioester bond.

17. The pharmaceutical composition of claim 15 or claim 16, wherein X is a chemical linker consisting of atoms selected from C, N, S, O, and/or H.

18. The pharmaceutical composition of any one of claims 15-17, wherein X is a chemical linker comprising between 1 and 8 carbon atoms.

19. The pharmaceutical composition of any one of claims 15-18, wherein X is a chemical linker comprising an alkylene chain, wherein, optionally, one or more carbon atoms of the alkylene chain being replaced with oxygen.

20. The pharmaceutical composition of any one of claims 11-19, wherein the dimer has the following structure:

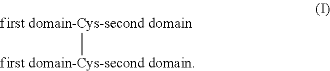

21. The pharmaceutical composition of any one of claims 1-20, wherein the first monomer and the second monomer each independently comprise an amino acid sequence that is at least 70% identical to the amino acid sequence of SEQ ID NOS:1, 2, 3, or 4.

22. The pharmaceutical composition of any one of claims 1-21, wherein the first monomer and the second monomer each independently comprise an amino acid sequence that is identical to the amino acid sequence of SEQ ID NOS:1, 2, 3, or 4.

23. The pharmaceutical composition of any one of claims 1-22, wherein the dimer comprises identical monomers.

24. The pharmaceutical composition of any one of claims 1-23, wherein the dimer comprises non-identical monomers and the ratio is calculated based on combined total of free monomer.

25. The pharmaceutical composition of claim 24, wherein the first monomer and the second monomer of the dimer have different C-terminal modifications.

26. The pharmaceutical composition of any one of claims 1-25, wherein the dimer has a structure selected from the group consisting of:

| | |
|---|---|
| GRKKRRQRRRCDLADNIERLKANDGLKFSQEYESI | SEQ ID NO: 1 |
| \| | |
| GRKKRRQRRRCDLADNIERLKANDGLKFSQEYESI | SEQ ID NO: 1 |
| GRKKRRQRRRCELADHIERLKANDNLKFSQEYESI | SEQ ID NO: 2 |
| \| | |
| GRKKRRQRRRCELADHIERLKANDNLKFSQEYESI | SEQ ID NO: 2 |
| GRKKRRQRRRCDMAEHTERLKANDSLKLSQEYESI | SEQ ID NO: 3 |
| \| | |
| GRKKRRQRRRCDMAEHTERLKANDSLKLSQEYESI | SEQ ID NO: 3 |
| GRKKRRQRRRCDMAEHMERLKANDSLKLSQEYESI | SEQ ID NO: 4 |
| \| | |
| GRKKRRQRRRCDMAEHMERLKANDSLKLSQEYESI | SEQ ID NO: 4 |

27. A method of repairing the nervous system and/or treating a neurological condition, disease or disorder selected from the group consisting of neural injury, neurological disease caused by inflammation or autoimmunity, and neurodegenerative disease, in a subject in need thereof, the method comprising administering an effective amount of the pharmaceutical composition of any one of claims 1-26 to a subject in need thereof.

28. The method of claim 27, wherein the neural injury is selected from the group consisting of acute neural injury, traumatic brain injury (TBI), spinal cord injury, concussion, stroke, including ischemic stroke, hemorrhagic stroke, and chronic stroke disease, aneurysm, cerebral hemorrhage, thrombus, and embolism.

29. The method of claim 27, wherein the neurological condition, disease or disorder is selected from the group consisting of Alzheimer's Disease, dementias related to Alzheimer's Disease, Lewy diffuse body diseases, senile dementia, Parkinson's Disease, amyotrophic lateral sclerosis, multiple sclerosis (MS), optic neuritis, Huntington's Disease, Tourette's syndrome, hereditary motor and sensory neuropathy, diabetic neuropathy, progressive supranuclear palsy, Jakob-Creutzfeldt disease, epilepsy, and infectious disease.

30. Use of the pharmaceutical composition of any one of claims 1-26 in the manufacture of a medicament for treatment of a neurological condition, disease or disorder selected from the group consisting of neural injury, neurological disease caused by inflammation or autoimmunity, and neurodegenerative disease.

31. A process for preparing the pharmaceutical composition of any one of claims 1-26, the process comprising combining the first monomer and the second monomer in water and either a) adding an oxidating agent and/or b) oxygenizing the solution, such that the dimer forms.

32. The process of claim 31, wherein the combined concentration of the first and second monomer in water is at least 20 mg/mL.

33. The process of claim 31 or claim 32, wherein the combined concentration of the first and second monomer in water is at least 40 mg/mL.

34. The process of any one of claims 31-33, wherein the combination of the first and second monomer in water is held at a temperature of about 20° C. to about 25° C.

35. The process of any one of claims 31-34, further comprising the addition of DMSO to the first and second monomers and water.

36. The process of any one of claims 31-35, wherein the oxidating agent is selected from among cupric sulfate, iodide, hydrogen peroxide, trans-3,4-dihydroxyselenolane oxide (DHS), supported methionine sulfoxide, and N-Chlorosuccinimide (NCS).

37. The process of any one of claims 31-36, wherein the oxidating agent is iodine (I2), and the process employs microwave-assisted oxidation.

38. A process for preparing the pharmaceutical composition of any one of claims 1-23 or 26, the process comprising combining the first monomer and the second monomer in a solvent with cupric sulfate to form a mixture to produce the dimer;
wherein the first monomer is identical to the second monomer.

39. The process of claim 38, wherein the solvent comprises purified water for purification (PWP).

40. The process of claim 39, wherein the solvent further comprises ethanol.

41. The process of any one of claims 38-40, wherein the pH of the mixture is maintained between about 8.5 and about 9.5.

42. A pharmaceutical agent for repairing the nervous system of a subject comprising a peptide dimer or pharmaceutically acceptable salt or solvate thereof, comprising two subunits, wherein each subunit comprises a peptide domain independently selected from receptor-type protein-tyrosine phosphatase (PTPR) wedge domains or variants having at least 70% homology thereto.

43. The pharmaceutical agent of claim 42, wherein each subunit comprises a transport moiety attached to the peptide domain via one cysteine residue or a peptide linker comprising one cysteine residue, wherein the two subunits are covalently cross-linked via a chemical linker between the cysteine residues on each subunit.

44. The pharmaceutical agent of claim 43, wherein the chemical linker is chosen from a disulfide bond, a thioether bond, or a thioester bond.

45. The pharmaceutical agent of claim 43, wherein the chemical linker comprises between 1 and 8 carbon atoms.

46. The pharmaceutical agent of any one of claims 42-45, wherein the peptide domain of each subunit independently comprises an amino acid sequence having at least 70% identity to the amino acid sequence of SEQ ID NO:8, 9, 10, or 11.

47. The pharmaceutical agent of any one of claims 43-46, wherein the transport moiety is chosen from a group consisting of an HIV TAT peptide, a herpes simplex virus-1 DNA binding protein VP22 peptide, an amino acid region of the third alpha-helix of antennapedia homeodomain, an histidine tag ranging in length from 4 to 30 histidine repeats, a variation derivative or homologue thereof capable of facilitating uptake of the active cargo moiety by a receptor independent process, a cationic arginine-rich peptide, and combinations thereof.

48. The pharmaceutical agent of any one of claims 43-47, wherein the transport moiety comprises an amino acid sequence having at least 65% identity to wild type HIV TAT.

49. The pharmaceutical agent of claim 48, wherein the TAT sequence comprises an amino acid sequence that is at least 65% identical to the amino acid sequence of SEQ ID NO:5, 6, or 7.

50. The pharmaceutical agent of any one of claims 42-49, wherein each subunit independently comprises an amino acid sequence that is at least 70% identical to the amino acid sequence of SEQ ID NOS:1, 2, 3, or 4.

51. The pharmaceutical agent of any one of claims 42-50, wherein the subunits have different C-terminal modifications.

52. The pharmaceutical agent of any one of claims 42-51, wherein the peptide dimer comprises two different subunits.

53. The pharmaceutical agent of any one of claims 42-51, wherein the peptide dimer comprises two identical monomer subunits.

54. The pharmaceutical agent of any one of claims 42-53, wherein the peptide dimer or pharmaceutically acceptable salt or solvate thereof has a purity of at least 90%.

55. A pharmaceutical composition comprising the pharmaceutical agent of any one of claims 42-54.

56. The pharmaceutical composition of claim 55, wherein the pharmaceutical composition does not comprise DMSO.

57. The pharmaceutical composition of any one of claims 1-26, which does not comprise DMSO.

58. Use of the pharmaceutical agent of any one of claims 42-54 in the manufacture of a medicament for repairing the nervous system and/or treatment of a neurological condition, disease or disorder selected from the group consisting of neural injury, neurological disease caused by inflammation or autoimmunity, and neurodegenerative disease.

59. A method of repairing the nervous system and/or treating a neurological condition, disease or disorder selected from the group consisting of neural injury, neurological disease caused by inflammation or autoimmunity, and neurodegenerative disease, in a subject in need thereof, the method comprising administering an effective amount of the pharmaceutical agent of any one of claims 42-54 to a subject in need thereof.


SEQUENCE LISTING (19 sequences)

SEQ ID NO: 1 - protein, 35 aa, synthetic construct
GRKKRRQRRRCDLADNIERLKANDGLKFSQEYESI

SEQ ID NO: 2 - protein, 35 aa, synthetic construct
GRKKRRQRRRCELADHIERLKANDNLKFSQEYESI

SEQ ID NO: 3 - protein, 35 aa, synthetic construct
GRKKRRQRRRCDMAEHTERLKANDSLKLSQEYESI

SEQ ID NO: 4 - protein, 35 aa, synthetic construct
GRKKRRQRRRCDMAEHMERLKANDSLKLSQEYESI

SEQ ID NO: 5 - protein, 10 aa, synthetic construct
GRKKRRQRRR

SEQ ID NO: 6 - protein, 11 aa, synthetic construct
YGRKKRRQRRR

SEQ ID NO: 7 - protein, 12 aa, synthetic construct
GRKKRRQRRRPQ

SEQ ID NO: 8 - protein, 24 aa, synthetic construct
DLADNIERLKANDGLKFSQEYESI

SEQ ID NO: 9 - protein, 24 aa, synthetic construct
ELADHIERLKANDNLKFSQEYESI

SEQ ID NO: 10 - protein, 24 aa, Homo sapiens
DMAEHTERLKANDSLKLSQEYESI

SEQ ID NO: 11 - protein, 24 aa, synthetic construct
DMAEHMERLKANDSLKLSQEYESI

SEQ ID NO: 12 - protein, 35 aa, Mus musculus
PIPITDLADNIERLICANDGICLFSQEYESIDPGQ

SEQ ID NO: 13 - protein, 34 aa, Rattus sp.
PIPITDLADNIERLICANDGKLFSQEYESIDPGQ

SEQ ID NO: 14 - protein, 34 aa, Homo sapiens; identical to SEQ ID NO: 13

SEQ ID NO: 15 - protein, 33 aa, Mus musculus
PIPITDMAEHMERLKANDSLKLSOEYESIDPGQ

SEQ ID NO: 16 - protein, 33 aa, Rattus sp.
PIPITDMAEHMERLKANDSLKLSQEYESIDPGQ

SEQ ID NO: 17 - protein, 33 aa, Homo sapiens
PIPIADMAEHTERLKANDSLKLSOEYESIDPGQ

SEQ ID NO: 18 - protein, 33 aa, Mus musculus
PIPILELADHIERLKANDNLKFSQEYESIDPGQ

SEQ ID NO: 19 - protein, 34 aa, Homo sapiens
PIPILELADHIERLKANDNLICFSOEYESIDPGQ